(19) United States
(12) Patent Application Publication
(10) Pub. No.: US 2024/0299171 A1
(43) Pub. Date: Sep. 12, 2024

(54) METHODS AND DEVICES FOR FOLDING VALVE LEAFLETS

(71) Applicant: EDWARDS LIFESCIENCES CORPORATION, Irvine, CA (US)

(72) Inventors: Tamir S. Levi, Zikhron Yaakov (IL); Ziv Yohanan, Kfar Hahoresh (IL); Assaf Bash, Benyamina-Givat Ada (IL)

(21) Appl. No.: 18/664,182

(22) Filed: May 14, 2024

Related U.S. Application Data
(63) Continuation of application No. PCT/US2022/049975, filed on Nov. 15, 2022.
(60) Provisional application No. 63/324,562, filed on Mar. 28, 2022.
(60) Provisional application No. 63/280,009, filed on Nov. 16, 2021.

(51) Int. Cl. A61F 2/24

(52) U.S. Cl. CPC A61F 2/2463; A61F 2/2418; A61F 2/2466; A61F 2210/0014; A61F 2220/0008; A61F 2220/0075; A61F 2250/0063; A61F 2250/0098

(57) ABSTRACT

A method of valve implantation includes installing a leaflet lasso on a host leaflet assembly at an implantation site within a patient's body. The method includes positioning an expandable prosthetic heart valve in a crimped state within a central region of the host leaflet assembly. The method includes radially expanding the prosthetic heart valve to a working diameter. During at least a portion of radially expanding the prosthetic heart valve, the prosthetic heart valve contacts a central lasso portion of the leaflet lasso and applies a radial force to the central lasso portion that results in folding of at least one leaflet of the leaflet assembly.

### CROSS-REFERENCE TO RELATED APPLICATIONS

The present application is a continuation of PCT/US2022/049975, filed Nov. 15, 2022, which claims the benefit of U.S. Provisional Patent Application No. 63/280,009, filed Nov. 16, 2021, and U.S. Provisional Patent Application No. 63/324,562, filed Mar. 28, 2022, all of which applications are incorporated by reference herein in their entirety.

### FIELD

The disclosure relates to methods and devices for reducing the risk of coronary artery ostia obstruction due to valve implantation.

### BACKGROUND

The human heart can suffer from various valvular diseases. These valvular diseases can result in significant malfunctioning of the heart that may ultimately require repair or replacement of the native valve with an artificial valve. There are a number of known repair devices (e.g., stents) and artificial valves and a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (e.g., through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

While implanting a guest valve within a host valve in both native TAVR and ViV TAVR, there is a risk of the guest valve displacing the leaflets of the host valve (either the native leaflets or the leaflets of a previously implanted prosthetic valve) outwards towards the coronary ostia. In some cases, the displacement can be such that a host leaflet partially or fully blocks a coronary ostium, causing coronary artery obstruction. The risk of coronary artery obstruction can be relatively higher in ViV TAVR compared to native TAVR.

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, the laceration or severing is performed prior to implantation of the guest valve, which carries a risk of blood leakage through the lacerated portion of the host leaflets until the guest valve can be implanted.

### SUMMARY

In one aspect, a leaflet lasso for modifying a leaflet of a host heart valve can be summarized as comprising a central lasso portion defining a central opening and at least one lateral lasso portion coupled to the central lasso portion such that radial expansion of the central lasso portion by applying tension to the central lasso portion results in axial contraction of the lateral lasso portion.

In another aspect, a leaflet lasso for modifying a leaflet of a host heart valve can be summarized as comprising a central lasso portion defining a central opening and a plurality of lateral lasso portions, each of the lateral lasso portions coupled to the central lasso portion such that radial expansion of the central lasso portion by applying tension to the central lasso portion results in axial contraction of the lateral lasso portion.

In another aspect, a prosthetic device can be summarized as comprising an annular frame movable between a radially compressed configuration and a radially expanded configuration, a leaflet assembly disposed within the annular frame and coupled to the annular frame, and a leaflet lasso. The leaflet lasso comprises a central lasso portion positioned over an end of the leaflet assembly, the central lasso portion defining a central opening and at least one lateral lasso portion fastened to a leaflet of the leaflet assembly. The lateral lasso portion is coupled to the central lasso portion such that radial expansion of the central lasso portion by applying tension to the central lasso portion results in axial contraction of the lateral lasso portion.

In yet another aspect, a prosthetic device can be summarized as comprising an annular frame movable between a radially compressed configuration and a radially expanded configuration, a leaflet assembly disposed within the annular frame and coupled to the annular frame, and a leaflet lasso. The leaflet lasso comprises a central lasso portion positioned over an end of the leaflet assembly, the central lasso portion defining a central opening and a plurality of lateral lasso portions fastened to a plurality of leaflets of the leaflet assembly. Each of the lateral lasso portions coupled to the central lasso portion such that radial expansion of the central lasso portion by applying tension to the central lasso portion results in axial contraction of each of the lateral lasso portions.

In another aspect, a method can be summarized as comprising positioning a central lasso portion of a leaflet lasso over an end of a leaflet assembly of a heart valve, attaching a lateral lasso portion of the leaflet lasso to a leaflet of the leaflet assembly, and applying tension to the central lasso portion to radially expand the central lasso portion. The lateral lasso portion axially contracts as the central lasso portion radially expands and folds the leaflet along an axial direction of the leaflet.

In another aspect, a method can be summarized as comprising installing a leaflet lasso on a leaflet assembly of a first prosthetic heart valve implanted within a native heart valve within a patient's body, positioning a second prosthetic heart valve in a crimped state within a central region of the leaflet assembly, and radially expanding the second prosthetic heart valve from the crimped state to a working diameter. During at least a portion of radially expanding the second prosthetic heart valve, the second prosthetic heart valve contacts a central lasso portion of the leaflet lasso and applies a radial force to the central lasso portion that results in folding of at least one leaflet of the leaflet assembly.

In another aspect, a leaflet lasso for modifying a leaflet of a host heart valve can be summarized as comprising a central lasso portion defining a central opening, at least one lateral lasso portion coupled to the central lasso portion such that radial expansion of the central lasso portion by applying tension to the central lasso portion results in axial contraction of the lateral lasso portion, and a tissue anchor comprising a shell member having a cavity and a core member disposed at least partially within the cavity. The lateral lasso portion comprises a first strand portion and a second strand portion. A first tail end of the first strand portion is coupled to the core member of the tissue anchor. The first tail end is releasable from the core member by axial movement of the core member relative to the shell member in a first direction.

In another aspect, a method can be summarized as comprising fastening a leaflet lasso to a leaflet assembly of a first prosthetic heart valve implanted within a native heart valve within a patient's body using a tissue anchor coupled to the leaflet lasso, positioning a second prosthetic heart valve in a crimped state within a central region of the leaflet assembly; radially expanding the second prosthetic heart valve from the crimped state to a working diameter, wherein during at least a portion of radially expanding the second prosthetic heart valve the second prosthetic heart valve contacts a central lasso portion of the leaflet lasso and applies a radial force to the central lasso portion that results in folding of a leaflet of the leaflet assembly; detaching the leaflet lasso from the leaflet assembly by applying tension to a release member coupled to the tissue anchor; and removing the leaflet lasso from the patient's body.

In another aspect, a medical assembly can be summarized as comprising a handle, a tube, a hollow needle, and an implantable leaflet folding device. The tube has a first proximal end, a first distal end, and a first lumen extending from the first proximal end to the first distal end. The tube is coupled to the handle and is axially displaceable relative to the handle. The hollow needle has a second proximal end, a second distal end, and a second lumen extending from the second proximal end to the second distal end. The hollow needle is disposed within the first lumen and is axially movable within the first lumen and relative to the tube. The implantable leaflet folding device is coupled to the hollow needle.

In another aspect, a delivery accessory for a leaflet folding device can be summarized as comprising a tube and a hollow needle. The tube has a first proximal end, a first distal end, and a first lumen extending from the first proximal end to the first distal end. The tube has a first longitudinal slot extending from the first distal end towards the first proximal end. The hollow needle has a second proximal end, a second distal end, and a second lumen extending from the second proximal end to the second distal end. The hollow needle is disposed within the first lumen and is axially movable within the first lumen and relative to the tube. The hollow needle has a second longitudinal slot extending from the second distal end towards the first proximal end. The second longitudinal slot is radially aligned with the first longitudinal slot to form a passage through which at least a portion of the leaflet folding device can extend into the second lumen.

In another aspect, a leaflet folding device for a host heart valve can be summarized as comprising a lasso member and an anchor member. The lasso member comprises a central lasso portion having a central axis and a lateral lasso portion connected to the central lasso portion and radially offset from the central axis. Radial expansion of the central lasso portion axially contracts the lateral lasso portion. The anchor member is coupled to the lasso member and comprises a first anchor radially offset from the central axis.

In another aspect, a leaflet folding device for a host heart valve can be summarized as comprising a curved member, an anchor member, and a tether. The curved member has a first end portion and a second end portion spaced from each other. The tether has a first tether portion extending between the first end portion and the second end portion and a second tether portion extending between the second end portion and the anchor member. The tether is slidably coupled to the second end portion such that radial expansion of the curved member increases a length of the first tether portion and correspondingly decreases a length of the second tether portion.

In another aspect, a leaflet folding device for a host heart valve can be summarized as comprising a guide element; a wire member, and an anchor member. The wire member is looped through the guide element to define a central lasso portion and a lateral lasso portion. A length of the central lasso portion and a length of the lateral lasso portion are mutually adjustable by sliding the wire member relative to the guide element. The anchor member is coupled to the lateral portion.

The foregoing general description and the following detailed description are exemplary of the claimed subject matter and are intended to provide an overview or framework for understanding the nature of the subject matter as it is claimed. The accompanying drawings are included to provide further understanding of the subject matter and are incorporated in and constitute a part of the specification.

### BRIEF DESCRIPTION OF THE DRAWINGS

The following is a description of the figures in the accompanying drawings. In the drawings, identical reference numbers identify similar elements or acts.

FIG. 1 is a perspective view of a prosthetic heart valve, according to one example.

FIG. 2 is a side elevation of the prosthetic heart valve of FIG. 1, depicted with the inner and outer skirts removed.

FIG. 3 illustrates a geometry of a leaflet, according to one example.

FIG. 4 illustrates delivery of a prosthetic heart valve to an aortic root, according to one example.

FIG. 5 is a cross-sectional view of a native aortic valve.

FIG. 6 illustrates implantation of a prosthetic heart valve within the native aortic valve of FIG. 5, according to one example.

FIG. 7 illustrates a valve-in-valve implantation within the native aortic valve of FIG. 5, according to one example.

FIG. 8 illustrates folding of the leaflets of the native aortic valve around the prosthetic heart valve, according to one example.

FIG. 9 is a perspective view of a leaflet lasso, depicted as attached to a leaflet assembly, according to one example.

FIG. 10 is a plan view of the leaflet lasso of FIG. 9.

FIG. 11 is a plan view of the leaflet lasso of FIG. 9, with tension applied to the central lasso portion of the leaflet lasso.

FIG. 12 illustrates positioning of a transcatheter apparatus at an implantation site for construction and attachment of the leaflet lasso.

FIGS. 13 and 14 illustrate construction and attachment of the leaflet lasso to leaflets of the native aortic valve.

FIG. 15 is a perspective view of the prosthetic heart valve, depicted with the leaflet lasso of FIG. 9.

FIG. 16 illustrates the prosthetic heart valve of FIG. 15 after tensioning the central lasso portion of the leaflet lasso to fold the leaflets of the valve.

FIG. 17 illustrates an expandable prosthetic heart valve in a crimped state extending through a leaflet lasso attached to leaflets of a host prosthetic heart valve.

FIG. 18 illustrates a valve-in-valve assembly formed by the expandable prosthetic heart valve and the host prosthetic heart valve of FIG. 17 and folding of the leaflets of the host prosthetic heart valve.

FIG. 19 is a perspective view of a leaflet lasso, depicted as attached to a leaflet assembly, according to another example.

FIG. 20 is an elevated view of a tissue anchor, according to one example.

FIGS. 21A-21E illustrate construction of the leaflet lasso of FIG. 19 using a tension member and tissue anchors.

FIG. 22 is a perspective view of a leaflet lasso, depicted as attached to a leaflet assembly, according to another example.

FIG. 23 is a perspective view of a leaflet lasso, according to the example of FIG. 22, having lateral lasso portions looped through respective leaflets of the leaflet assembly.

FIG. 24 is a perspective view of the prosthetic heart valve, depicted with the leaflet lasso of FIG. 22.

FIG. 25 is a perspective view of a leaflet lasso, depicted as attached to a leaflet assembly, according to another example.

FIG. 26 is a perspective view of a leaflet lasso having an interwound portion, according to another example.

FIG. 27 is a perspective view of a leaflet lasso having a lateral lasso portion looped through a leaflet of a leaflet assembly, according to another example.

FIG. 28 is a perspective view of a leaflet lasso having a central lasso portion with multiple windings, according to another example.

FIG. 29 is a perspective view of the prosthetic heart valve, depicted with the leaflet lasso of FIG. 26.

FIG. 30 illustrates a lasso anchor system, according to one example.

FIG. 31 illustrates a leaflet lasso attached to host leaflets using the lasso anchor system of FIG. 30.

FIG. 32 illustrates the lasso anchor system of FIG. 30 with a strand portion of a lateral lasso portion detached from a tissue anchor of the lasso anchor system.

FIG. 33 illustrates a leaflet lasso attached to host leaflets using the lasso anchor system of FIG. 30 with a strand portion of a leaflet lasso portion detached from a tissue anchor of the lasso anchor system.

FIG. 34 illustrates a leaflet lasso attached to host leaflets using a plurality of the lasso anchor system of FIG. 30.

FIG. 35A illustrates a lasso anchor system, according to another example.

FIG. 35B is detail view of circled region 35B of FIG. 35A.

FIG. 36 illustrates the lasso anchor system of FIG. 35A with a strand portion of a lateral lasso portion detached from a tissue anchor of the lasso anchor system.

FIG. 37 illustrates an implantable leaflet folding device, according to another example.

FIG. 38 illustrates the leaflet folding device of FIG. 37 coupled to a delivery accessory, according to one example.

FIGS. 39A and 39B illustrate the delivery accessory of FIG. 38 and the leaflet folding device of FIG. 37 coupled to a delivery apparatus, according to one example.

FIGS. 40A and 40B illustrate the delivery accessory of FIG. 38 being used to implant the leaflet folding device on a leaflet of a host valve.

FIG. 41 is a perspective view of a prosthetic heart valve, depicted with the leaflet folding device of FIG. 37.

FIG. 42 illustrates a leaflet folding device that incorporates a flexible tether, such as a suture, according to another example.

FIG. 43 illustrates a leaflet folding device made from a continuous wire, according to another example.

FIG. 44 is a perspective view of a prosthetic heart valve, depicted with the leaflet folding device of FIG. 43.

### DETAILED DESCRIPTION

The subject matter is described with implementations and examples. In some cases, as will be recognized by one skilled in the art, the disclosed implementations and examples may be practiced without one or more of the disclosed specific details, or may be practiced with other methods, structures, and materials not specifically disclosed herein. All the implementations and examples described herein and shown in the drawings may be combined without any restrictions to form any number of combinations, unless the context clearly dictates otherwise, such as if the proposed combination involves elements that are incompatible or mutually exclusive. The sequential order of the acts in any process described herein may be rearranged, unless the context clearly dictates otherwise, such as if one act requires the result of another act as input.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

The word “comprise” and derivatives thereof, such as “comprises” and “comprising”, are to be construed in an open, inclusive sense, that is, as “including, but not limited to”. The singular forms “a”, “an”, “at least one”, and “the” include plural referents, unless the context dictates otherwise. The term “and/or”, when used between the last two elements of a list of elements, means any one or more of the listed elements. The term “or” is generally employed in its broadest sense, that is, as meaning “and/or”, unless the context clearly dictates otherwise.

The term “coupled” without a qualifier generally means physically coupled or linked and does not exclude the presence of intermediate elements between the coupled elements absent specific contrary language. The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus.

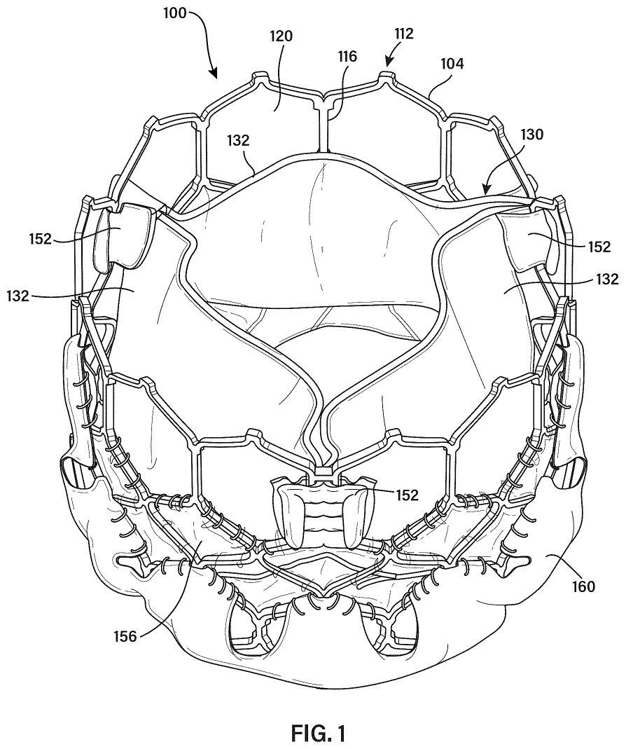

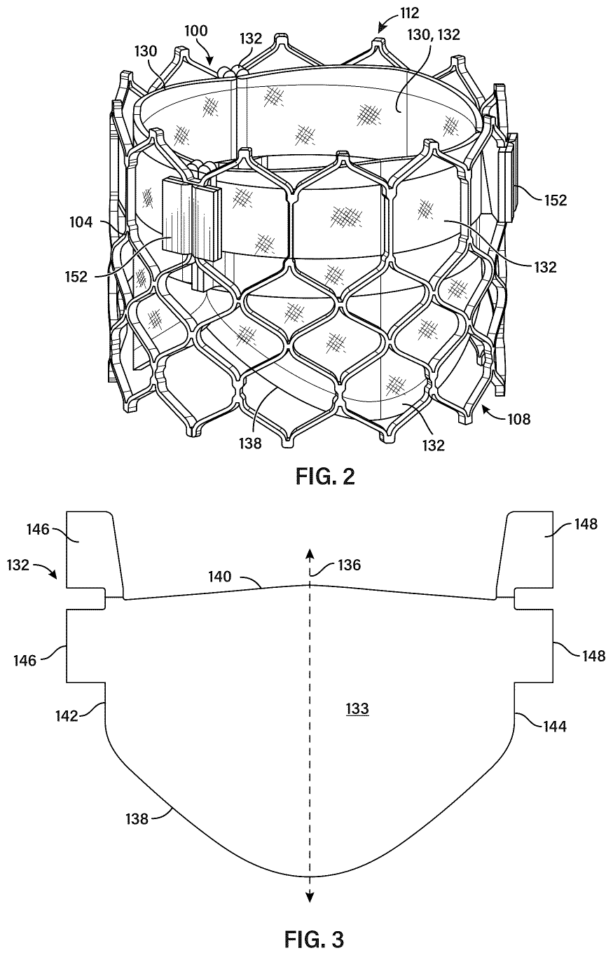

FIGS. 1 and 2 illustrate an exemplary prosthetic heart valve 100 that can be delivered to an implantation site within a patient's body. The prosthetic heart valve 100 is adjustable between a radially compressed configuration (or crimped state) and a radially expanded configuration. The prosthetic heart valve 100 can be positioned in the crimped state for ease of delivery to the implantation site via the patient's vasculature and then expanded to working diameter at the implantation site. One use of the prosthetic heart valve 100 is as a replacement aortic valve. Other uses of the prosthetic heart valve 100 can be as a replacement mitral valve, a replacement pulmonary valve, or a replacement tricuspid valve. The prosthetic heart valve 100 can be implanted within a native valve or within a prosthetic valve previously implanted within a native valve.

The prosthetic heart valve 100 can include a frame 104 (or stent) with an annular shape. The frame 104 can have an inflow end 108 (shown in FIG. 2) and an outflow end 112 (the terms “inflow” and “outflow” are relative to the direction of blood flow through the prosthetic heart valve when the prosthetic heart valve is implanted). In the illustrated example, the frame 104 includes a plurality of struts 116 that are interconnected to define the annular shape of the frame and a plurality of cells 120. The struts 116 of the frame 104 can be configured to allow the prosthetic heart valve 100 to be adjustable between the crimped state and the radially expanded configuration. The frame 104 is, however, not limited to the particular arrangement and shapes of the struts 116 shown in FIGS. 1 and 2.

In one example, the frame 104 can be a plastically-expandable frame made of a plastically-expandable material (e.g., stainless steel, a biocompatible high-strength alloy such as a cobalt chromium alloy or a nickel-cobalt chromium alloy, a polymer, or combinations thereof). The plastically-expandable frame can be radially expanded by an inflatable balloon (or other type of expandable member). In another example, the frame 104 can be a self-expanding frame made of a self-expanding material (e.g., nickel titanium alloy (NiTi), such as Nitinol). Further examples of plastically-expandable and self-expanding frames are described in, for example, U.S. Pat. No. 9,393,110 and U.S. Patent Publication No. 2018/0028310, which are incorporated herein by reference.

Alternatively, the prosthetic heart valve 100 can use a frame that is expandable by mechanical actuators. The mechanically expandable frame can have struts that are interconnected by pivoting joints. The mechanical actuators can be configured to pivot or bend the struts in order to move the frame between a radially compressed configuration and a radially expanded configuration. Examples of frames with pivoting joints are described in, for example, U.S. Patent Publication Nos. 2018/0153689, 2018/0344456, 2019/0060057, and 2019/0105153, and U.S. patent application Ser. No. 16/788,090, filed Feb. 11, 2020, and 62/945,000, filed Dec. 6, 2019, all of which are incorporated herein by reference.

The prosthetic heart valve 100 can include a leaflet assembly 130 (or valvular structure) mounted within the frame 104. The leaflet assembly 130 can include one or more leaflets 132 that cycle through open and closed positions to regulate flow of blood through the prosthetic heart valve. In the example illustrated in FIGS. 1 and 2, the leaflet assembly 130 includes three leaflets 132. However, the leaflet assembly 130 can have fewer than three leaflets in other cases. Each leaflet 132 can be made wholly or partly from biological material, bio-compatible synthetic materials, or other such materials. Suitable biological material can include, for example, bovine pericardium (or pericardium from other sources).

FIG. 3 illustrates an example geometry of a leaflet 132. As illustrated, the leaflet 132 can have a leaflet body (or cusp) 133 and an axial axis 136. The leaflet body 133 can have a cusp edge 138 and a free edge 140 disposed generally opposite to the cusp edge 138. The free edge 140 can be transverse to the axial axis 136. The leaflet body 133 can have opposite side edges 142, 144, which can extend generally parallel to the axial axis 136. The cusp edge 138 can be coupled to the frame 104 when the leaflet assembly (130 in FIGS. 1 and 2) is mounted within the frame. The side edges 142, 144 can include tabs 146, 148 that can be used to form commissures. The free edge 140 can move towards and away from a center of the valve and can coapt with the free edges of other leaflets to close the valve.

Returning to FIGS. 1 and 2, the leaflet assembly 130 can be coupled to the frame 104 via commissures 152 (e.g., commissures formed with tabs on the side edges of the leaflets 132). Further details regarding the manner in which the leaflet assembly can be mounted to the frame can be found, for example, in U.S. Pat. Nos. 6,730,118; 7,393,360; 7,510,575; 7,993,394; 8,652,202; and 9,393,110; U.S. Patent Publication Nos. 2018/0028310, 2018/0325665, 2019/0105153, and 2019/0192296, U.S. Patent Application Nos. 62/797,837 (filed Jan. 28, 2019), 62/823,905 (filed Mar. 26, 2019), 62/854,702 (filed May 30, 2019), 62/928,993 (filed Oct. 31, 2019), 62/959,723 (filed Jan. 10, 2020), 62/971,011 (filed Feb. 6, 2020), 62/985,558 (filed Mar. 5, 2020), and 62/960,838 (filed Jan. 14, 2020); and International Patent Application Nos. PCT/US2019/61392 (filed Nov. 14, 2019) and PCT/US2020/18664 (filed Feb. 18, 2020), all of which are incorporated herein by reference in their entireties.

The prosthetic heart valve 100 can include one or more skirts or sealing members. For example, as illustrated in FIG. 1, the prosthetic heart valve 100 can include an inner skirt 156 mounted on an inner surface of the frame 104 and/or an outer skirt 160 mounted on an outer surface of the frame 104. The inner skirt 156 can be a circumferential inner skirt that spans an entire circumference of the inner surface of the frame 104. The inner skirt 156 can function as a sealing member to prevent or decrease paravalvular leakage (e.g., when the prosthetic heart valve is placed at the implantation site) and can be used to couple the inflow edges of the leaflets 132 to the frame 104. The outer skirt 160 can function as a sealing member by sealing against the tissue of the native valve annulus and helping to reduce paravalvular leakage past the prosthetic heart valve. In some cases, portions of a single skirt can provide the inner skirt 156 and the outer skirt 160.

The skirts 156, 160 can be formed from any of various suitable biocompatible materials, including any of various synthetic materials (e.g., polyethylene terephthalate (PET)) or natural tissue (e.g., pericardial tissue). The skirts 156, 160 can be mounted to the frame 104 using sutures, adhesive, welding, and/or other means for attaching the skirts to the frame. In some cases, the inner skirt 156 can serve as an attachment surface to anchor a portion of the leaflets 132 to the frame 104. For example, the inflow or cusp edge portions (138 in FIGS. 2 and 3) of the leaflets 132 can be sutured to the inner skirt 156, which can in turn be sutured to selected struts 116 of the frame 104. Further details regarding the skirts, techniques for assembling the leaflets to the inner skirt, and techniques for assembling the skirts on the frame are disclosed in U.S. Patent Application Publication Nos. 2012/0123529, 2019/0192296, and 2019/0365530, and International Patent Application Nos. PCT/US2020/014701 and PCT/US2020/024559, each of which is incorporated herein by reference.

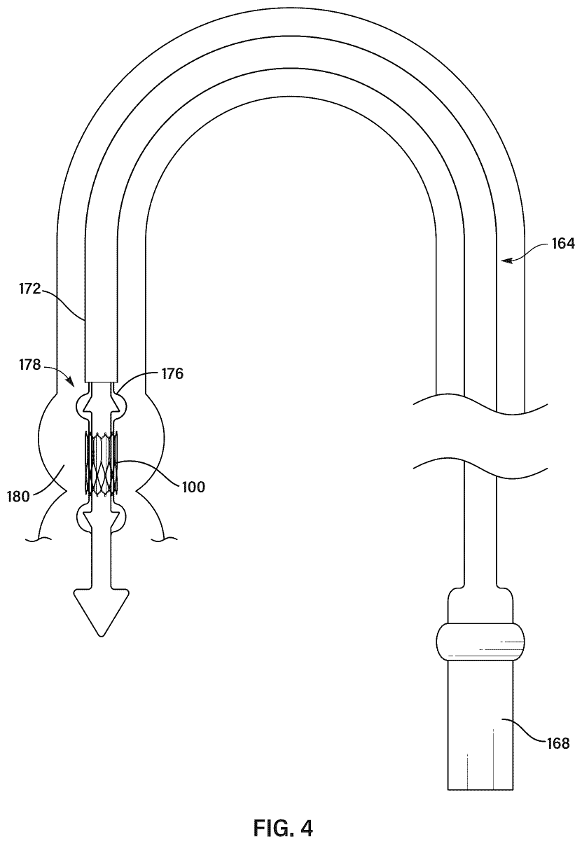

FIG. 4 illustrates a delivery apparatus 164 that can be used to deliver and implant a prosthetic valve. In one example, the delivery apparatus 164 is configured to deliver a plastically-expandable prosthetic valve. For example, the delivery apparatus 164 can include a handle 168 and an elongated shaft 172 coupled to the handle. The elongated shaft 172 can be advanced through a patient's vasculature while the handle 168 stays on the outside of the patient's body. The delivery apparatus 164 can further include an inflatable balloon 176 (which can be replaced with a different type of expansion member in other examples) extending distally from the elongated shaft 172 and operable by the handle 168. In one example, the prosthetic heart valve 100 can be mounted on the inflatable balloon 176 for delivery to an implantation site 180.

In another example, the delivery apparatus can be configured to deliver a self-expanding prosthetic valve. Further details of delivery apparatuses that can be used to deliver a plastically-expandable prosthetic valve are disclosed in, for example, U.S. Pat. No. 9,339,384 and U.S. Patent Publication Nos. 2017/0065415, 2016/0158497, and 2013/0030519, which are incorporated herein by reference. Further details of delivery apparatuses that can be used to deliver a self-expandable prosthetic valve are disclosed in, for example, U.S. Pat. Nos. 9,867,700 and 8,652,202, which are incorporated herein by reference. In other examples, the delivery apparatus can be configured to deliver a mechanically-expandable prosthetic valve. Examples of delivery apparatuses that can be used to deliver a mechanically-expandable prosthetic valve are disclosed in, for example, U.S. Pat. Nos. 9,827,093 and 10,806,573, which are incorporated herein by reference.

Initially, the prosthetic heart valve 100 can be mounted on the inflatable balloon 176 in a crimped state, as shown in FIG. 4. For aortic valve replacement, the distal end portion 178 of the delivery apparatus 164, which includes the prosthetic heart valve 100, can be inserted into a femoral artery and advanced through the femoral artery and the aorta to the implantation site 180. In some cases, a guidewire can be initially extended through the femoral artery and aorta. The guidewire can be subsequently used to guide the distal end portion 178 of the delivery apparatus 164 to the implantation site and to position the prosthetic heart valve 100 within a native aortic valve (or within an artificial valve previously implanted within the native aortic valve). After delivery of the prosthetic heart valve 100 to the implantation site, the inflatable balloon 176 can be inflated to expand the prosthetic heart valve 100 to a working diameter.

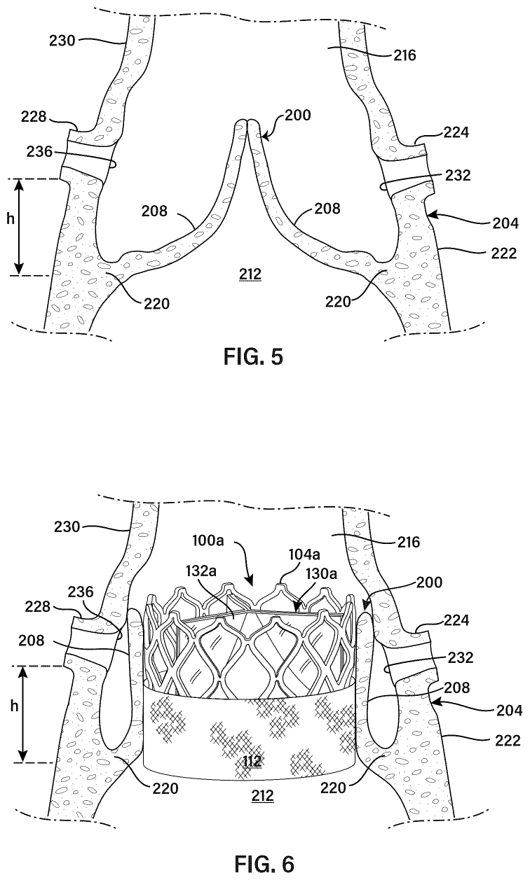

FIG. 5 illustrates an anatomy of the aortic root 204, which is positioned between the left ventricle 212 and the ascending aorta 216. The aortic root 204 includes a native aortic valve 200 having a plurality of leaflets 208. Normally, the native aortic valve 200 has three leaflets (only two leaflets are visible in the simplified illustration of FIG. 5), but aortic valves with fewer than three leaflets are possible. The leaflets 208 are supported by the aortic annulus 220, which is a ring of fibrous tissue at the transition point between the left ventricle 212 and the aortic root 204. The leaflets 208 can cycle between open and closed positions (the closed position is shown in FIG. 5) to regulate flow of blood from the left ventricle 212 to the ascending aorta 216.

Branching off the aortic root 204 are the coronary arteries 224, 228. The coronary artery ostia 232, 236 are the openings that connect the aortic root 204 to the coronary arteries 224, 228. Each of the coronary artery ostia 232, 236 can have an ostial height h, which can be defined as a distance from the coronary ostium 232, 236 to the aortic annulus 220. The ostial height of the coronary artery ostia 232, 236 are typically different and can be an important parameter when setting a replacement valve within the orifice of the native aortic valve 200.

FIG. 6 illustrates a hypothetical coronary artery obstruction that could occur in some cases from implantation of a prosthetic heart valve 100a within the native aortic valve 200. In this example, the prosthetic heart valve 100a is the guest valve or new valve, and the native aortic valve 200 is the host valve or old valve. The prosthetic heart valve 100a can have the general structure of the prosthetic heart valve 100 described with reference to FIGS. 1-3. For example, the prosthetic heart valve 100a can include an annular frame 104a and a leaflet assembly 130a mounted within the frame 104a. The leaflet assembly 130a can include flow regulating leaflets 132a.

During implantation of the prosthetic heart valve 100a, the prosthetic heart valve 100a is positioned within a central region defined between the leaflets 208 (“host leaflets”) of the native aortic valve 200 and radially expanded against the host leaflets 208. As illustrated, the host leaflets 208 form a tube around the frame 104a of the prosthetic heart valve 100a after the prosthetic heart valve 100a is radially expanded to the working diameter. As further illustrated, expansion of the prosthetic heart valve 100a displaces the host leaflets 208 outwards towards the coronary ostia 232, 236 such that the host leaflets 208 contact a portion of the aortic root 204 surrounding the coronary ostia 232, 236, causing coronary artery obstruction.

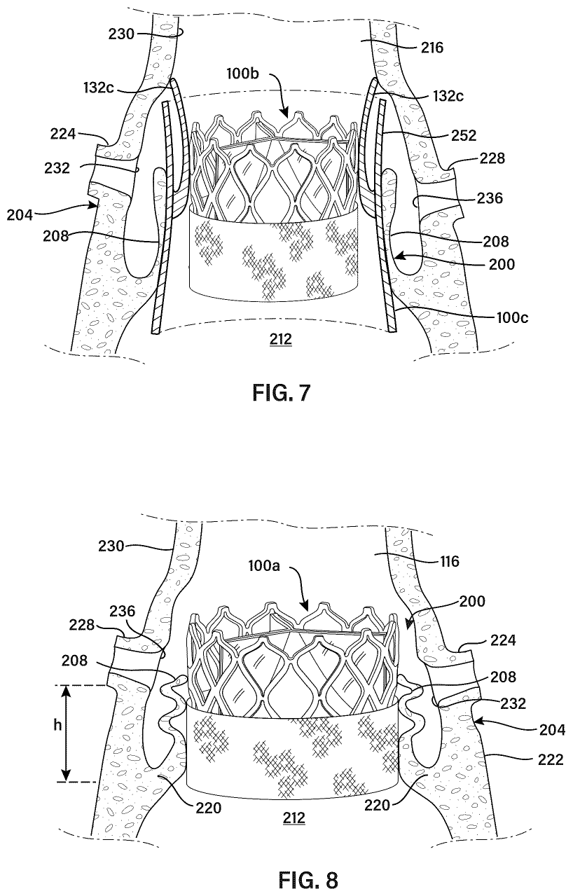

FIG. 7 illustrates another hypothetical coronary artery obstruction that could occur in some cases from implantation of a prosthetic heart valve 100b within a previously implanted prosthetic heart valve 100c (e.g., after a ViV procedure). In this example, the prosthetic heart valve 100b is the guest valve or new valve, and the prosthetic heart valve 100c is the host valve or old valve. In this example, the prosthetic heart valve 100c was previously implanted within the orifice of the native aortic valve 200. Each of the prosthetic heart valves 100b, 100c can have the general structure of the prosthetic heart valve 100 described with reference to FIGS. 1-3.

During implantation of the prosthetic heart valve 100b, the prosthetic heart valve 100b is positioned within a central region defined between the leaflets 132c (“host leaflets”) of the prosthetic heart valve 100c and radially expanded against the host leaflets 132c. As illustrated, the radial expansion of the prosthetic heart valve 100c results in outward displacement of the host leaflets 132c. As further illustrated, the host leaflets 132c are displaced such that the host leaflets 132c contact the aortic root 204 at positions superior to the coronary artery ostia 232, 236, causing coronary artery ostia obstruction. Alternatively, the prosthetic heart valve (or guest valve) 100b can displace the host leaflets 132c outwardly against the frame 252 of the prosthetic heart valve (or host valve) 100c, thereby blocking the flow of blood through the frame 252 to the coronary ostia 232, 236.

The risk illustrated in FIG. 7 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 6 and 7, the host leaflets are shown obstructing both coronary artery ostia 232, 236. In some cases, only one host leaflet may obstruct a respective coronary artery ostium. For example, the risk of obstructing the left coronary ostium 232 tends to be greater than obstructing the right coronary ostium 236 because the left coronary ostium 232 typically sits lower than the right coronary ostium 236.

A leaflet lasso configured for attachment to host leaflets prior to implanting a guest valve within the host valve is disclosed herein. Tension applied to the leaflet lasso results in folding of the host leaflets and/or downward displacement of the host leaflets, thereby preventing or reducing outward displacement of the host leaflets that could result in coronary artery obstruction in some cases. The guest valve can be an expandable prosthetic heart valve, such as the prosthetic valve 100 shown in FIGS. 1 and 2. In some cases, the force used to expand the guest valve to a working diameter after delivery of the guest valve to the host valve can be translated to tension applied to the leaflet lasso, allowing simultaneous expansion of the guest valve and folding of the host leaflets. For illustration purposes, FIG. 8 shows how folding the host leaflets 208 (previously illustrated in FIG. 6) can avoid coronary artery obstruction.

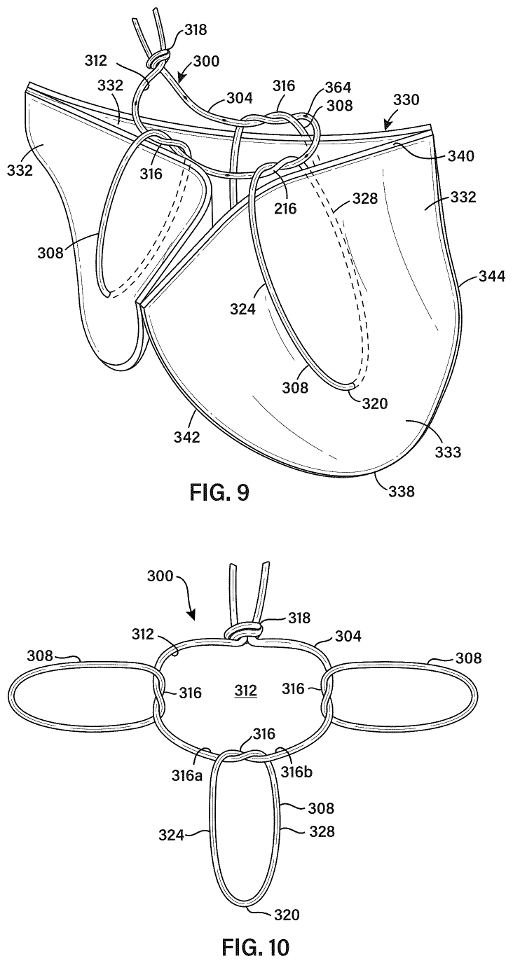

FIG. 9 illustrates an exemplary leaflet lasso 300 (also referred to herein as a leaflet lasso device or leaflet folding device), according to one implementation. For illustration purposes, the leaflet lasso 300 is depicted as attached to a representative leaflet assembly 330, which can be the leaflet assembly of a native heart valve or the leaflet assembly of an artificial valve (such as the prosthetic heart valve 100 shown in FIGS. 1 and 2).

The leaflet assembly 330 includes one or more leaflets 332 (three leaflets are shown for illustrative purposes). Each of the leaflets 332 has a cusp edge 338, a free edge 340, and opposite side edges 342, 344. The free edges 340 of the leaflets 332 are the edges of the leaflets 332 that can come together (or “coapt”) to close a valve. The cusp edge 338 can be attached to a frame (in the case of a prosthetic heart valve) or to an annulus (in the case of a native heart valve). Although not shown, the side edges 342, 344 of adjacent host leaflets 332 can be attached together (e.g., by suturing). Although not shown, the side edges 342, 344 of each host leaflet 332 can have tabs (as illustrated, for example, in FIG. 3) for forming commissures that attach the leaflet assembly to a frame (in the case of a prosthetic heart valve).

The leaflet lasso 300 includes a central lasso portion 304 and one or more lateral lasso portions 308. In the illustrated example, three lateral lasso portions 308 are shown, corresponding to the three leaflets 332 of the example leaflet assembly 330. In other examples, the number of lateral lasso portions 308 can be greater than or less than the number of leaflets in the leaflet assembly 330.

The central lasso portion 304 can form a closed loop and define a central opening 312. The leaflet lasso 300 can be formed from a piece of string having extreme end portions fastened together by a lock knot 318 (e.g., a double knot or square knot). The lock knot 318 can be considered as part of the central lasso portion 304.

Each lateral lasso portion 308 can form a closed loop. The loops formed by the lateral lasso portions 308 can be coupled to the loop formed by the central lasso portion 304 via slip knots 316 (or sliding knot, e.g., a half knot or single knot) that allow the central lasso portion 304 and the lateral lasso portions 308 to be mutually adjustable (i.e., an adjustment of the loop size of the central lasso portion 304 results in adjustment of the loop sizes of the lateral lasso portions 308, and vice versa).

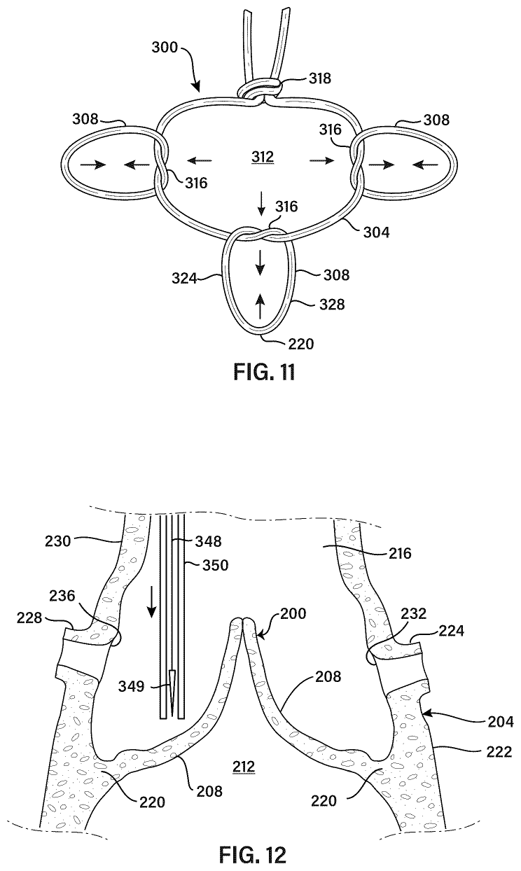

As illustrated more clearly in FIG. 10, each lateral lasso portion 308 can include two strand portions 324, 328. The lateral lasso portion 308 can further include an anchor portion 320 extending between the two strand portions 324, 328. The anchor portion 320 can have a U-shape or V-shape, for example. Each of the strand portions 324, 328 can tie one leg of the anchor portion 320 to the central lasso portion 304. At the junction between the central lasso portion 304 and each lateral lasso portion 308, the strand portions 324, 328 are looped over each other to form one of the slip knots 316 (e.g., a half knot or single knot). The portions of the strand portions 324, 328 extending past the slip knot 316 are connected to, or form part of, the central lasso portion 304, allowing the central lasso portion 304 and lateral lasso portion 308 to be mutually adjustable.

Returning to FIG. 9, the leaflet lasso 300 can be attached to the leaflet assembly 330 by positioning the central lasso portion 304 over an end of the leaflet assembly 330 including the free edges 340 of the leaflets 332 and by fastening the lateral lasso portions 308 to the leaflets 332. In the illustrated example, each lateral lasso portion 308 can be fastened to a respective leaflet 332 by extending the anchor portion 320 of the lateral loop 308 through a leaflet 332, such as through the leaflet at a location adjacent the cusp edge 338. The anchor portion 320 desirably is placed as close to the cusp edge as possible to maximize folding of the leaflets 332 and/or downward displacement of the leaflets 332 when a guest valve is implanted. The strand portions 324, 328 of each lateral lasso portion 308 are thereby positioned on opposite sides of the respective leaflet 332. The strand portions 324, 328 extend axially along the leaflet 332, placing the slip knot 316 formed between the strand portions 324, 328 at or near the free edge 340 of the leaflet 332.

Tension can be applied to the central lasso portion 304 to radially expand the central lasso portion 304. As illustrated in FIG. 11, as the central lasso portion 304 is radially expanded, the central lasso portion 304 will take up material from the strand portions 324, 328 of the lateral lasso portions 308 via the slip knots 316, reducing the length of the strand portions 324, 328 and axially contracting (or shortening) the lateral lasso portions 308. The lateral lasso portions 308 may contract by the same amount or by different amounts, depending on the uniformity of the expansion force acting along the central lasso portion 304. When the lateral lasso portions 308 are attached to the leaflets 332 as shown in FIG. 9, axial contraction of the lateral lasso portions 308 results in folding and downward displacement of the leaflets 332 in the axial direction of the leaflets.

The leaflet lasso 300 can be constructed from a tension member that is flexible and desirably non-elastic or substantially non-elastic to resist stretching when tension is applied. The tension member can be a suture, wire, cable, string, cord, yarn, or similar materials. The tension member can comprise one or more filaments. Multiple filaments can be spun or braided together, for example, to form a reinforced tension member. The tension member can be made of any of various suitable materials, such as natural fibers, synthetic fibers, plastics, and/or metals, to name a few. In one specific implantation, the tension member comprises a braided suture, such as Ethibond suture. The braided suture can be, for example, a 2-0, 3-0, 4-0, or 5-0 gauge suture.

The tension member can be initially free of knots. The lateral lasso portions 308 and the slip knots 316 of the leaflet lasso 300 can be formed in spaced portions of the tension member, as shown in FIGS. 10 and 11. The tail ends of the tension member can be secured together by a lock knot 318 to close the loop of the central lasso portion 304. In one example, the leaflet lasso 300 can be both constructed and attached to host leaflets within a patient's body.

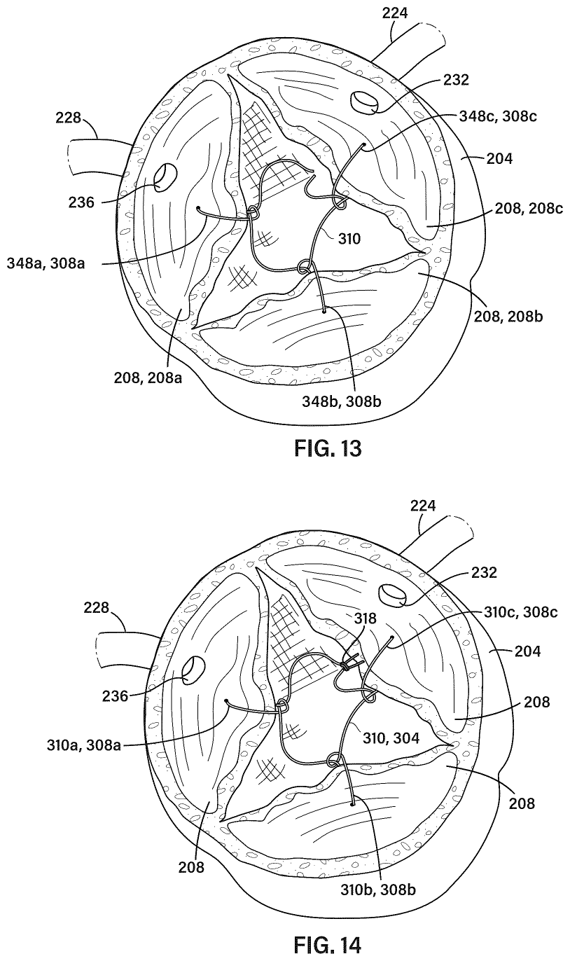

FIGS. 12-14 illustrate an example of constructing and attaching the leaflet lasso 300 (shown in FIGS. 9-11) to the leaflets 208 of the native aortic valve 200. The process can include attaching a tension member 348 to a needle 349 and guiding the tension member 348 and needle 349 to the location of the leaflets 208 using a suture device 350, which can be a transcatheter apparatus. Various examples of suturing devices are described in, for example, U.S. Patent Application No. 63/211,254 (filed Jun. 16, 2021), which is incorporated herein by reference.

Using the needle 349, a first portion 348a of the tension member can be looped through a first leaflet 208a, followed by forming a slip knot in the first portion 348a to complete a first lateral lasso portion 308a of the leaflet lasso. A second portion 348b of the tension member can be looped through a second leaflet 208b, followed by forming a slip knot to complete a second lateral lasso portion 308b of the leaflet lasso. A third portion 348c of the tension member can be looped through a third leaflet 208c, followed by forming a slip knot to complete a third lateral lasso portion 308c of the leaflet lasso. Finally, the extreme end portions of the tension 348 can be secured together with a locking knot to complete the central lasso portion 304 of the leaflet lasso.

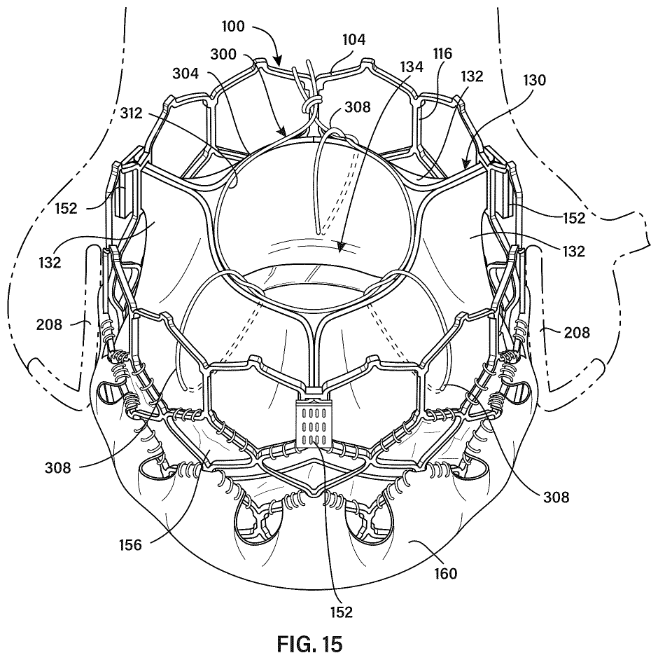

The process illustrated in FIGS. 12-14 can be used similarly to attach the leaflet lasso 300 (shown in FIGS. 9-11) to a prosthetic heart valve that is implanted within a native heart valve. For example, FIG. 15 shows the leaflet lasso 300 attached to the leaflet assembly 130 of the prosthetic heart valve 100 (which is depicted as implanted within a native heart valve). The central lasso portion 304 extends over the free edges of the leaflets 132. The lateral lasso portions 308 are fastened to the leaflets 132 (e.g., by looping the lateral lasso portions 308 through the leaflets). The central opening 312 defined by the central lasso portion 304 can be aligned with a central region 134 of the leaflet assembly 130 located between the leaflets 132. The central opening 312 can have an initial size to receive an expansion member (such as an inflatable balloon or an expandable valve).

In some examples, the leaflet lasso 300 can be pre-assembled on the prosthetic heart valve 100 before it is implanted within a patient. Once implanted, the lateral lasso portions 308 are initially in a relatively loose or slackened state, which allow the leaflets 132 to open and close under the flow of blood in a normal manner throughout the useful life of the prosthetic valve 100 (e.g., 10-20 years). Once the prosthetic valve 100 reaches the end of its useful life and a ViV procedure is to be performed, a guest prosthetic valve can be implanted within the prosthetic valve 100, as further described below. In alternative examples, the leaflet lasso 300 can be connected to the leaflets 132 after the prosthetic valve 100 is implanted and before performing the ViV procedure, using transcatheter suturing devices, as described above.

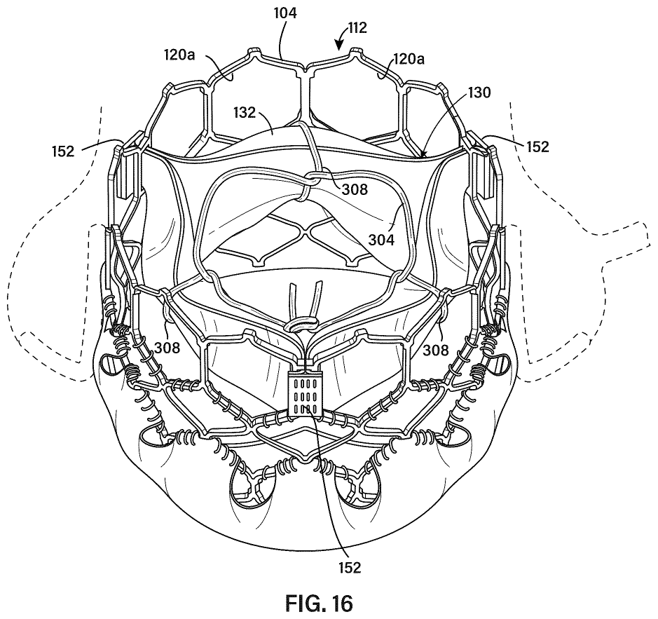

FIG. 16 shows the leaflet assembly 130 after applying some tension to the central lasso portion 304 to radially expand the central lasso portion 304. As shown, radial expansion of the central lasso portion 304 results in contraction of the lateral lasso portions 308, which in turn results in folding of the leaflets 132 of the leaflet assembly 130 towards the inflow end of the frame 104.

It can be observed in FIG. 16 that the folded leaflets 132 are anchored to the frame 104 at the commissures 152 and that the folded leaflets 132 have saddle points at the locations of the lateral lasso portions 308. These saddle points are below the commissure plane (i.e., a plane perpendicular to the central longitudinal axis and intersecting the commissures 152), which generally means that the folded leaflets 132 will stay between the commissure plane and the inflow end (bottom end in the orientation of the drawing) of the frame 104. In some examples, the saddle points of the leaflets 132 are at least below the outflow edges of the commissures 152. In some examples, the saddle points are at least below the inflow edges of the commissures. Thus, not only will the folded leaflets 132 not extend out from the outflow end (top end in the orientation of the drawing) of the frame 104, the cells 120a in the upper row (i.e., the row at the outflow end) of the frame 104 remain at least partially uncovered by the folded leaflets 132 to allow blood to flow through the cells 120a toward the coronary ostia.

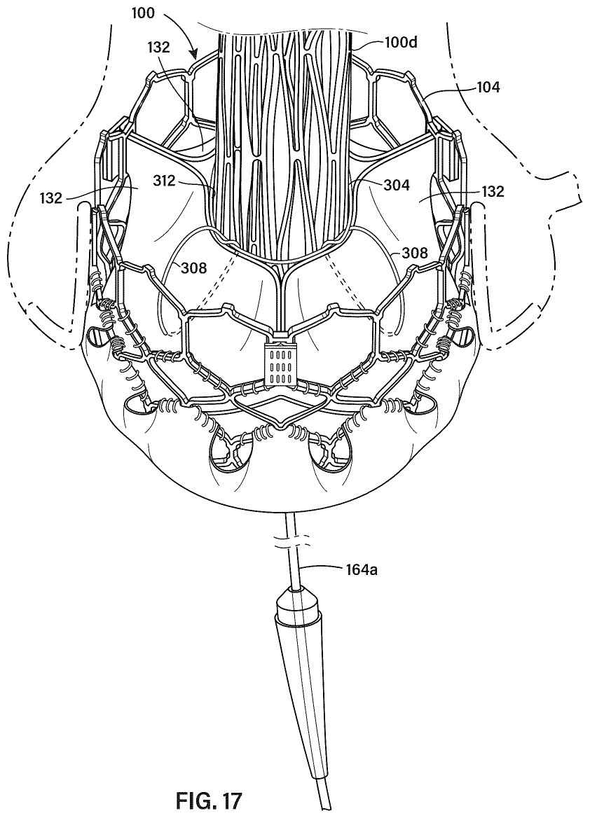

In one example, tension can be applied to the central lasso portion 304 by an expandable prosthetic heart valve (“guest valve”) to be implanted within the host valve. Initially, the leaflet lasso 300 can be attached to the leaflet assembly 130 of the prosthetic heart valve 100 (“host valve”) as shown in FIG. 15. As noted above, the leaflet lasso 300 can be pre-assembled on the prosthetic heart valve 100 prior to implantation or after implantation. As illustrated in FIG. 17, a guest valve 100d (e.g., an expandable heart valve) can be mounted on a distal end portion of a delivery apparatus 164a in a crimped state. The distal end portion of the delivery apparatus can be advanced to the host valve 100 to position the guest valve 100d within the central region (134 in FIG. 15) formed between the host leaflets 132. In this case, the initial size of the central opening 312 of the central lasso portion 304 can be selected to be greater than the outer diameter of the guest valve 100d in the crimped state.

Once the guest valve 100d is positioned in the central region defined between the host leaflets 132, the guest valve 100d can be radially expanded (e.g., by an inflatable balloon, by mechanical actuator(s), and/or by removing a restraining capsule or device from the guest valve). In addition to selecting the initial size of the central opening 312 to be greater than the outer diameter of the guest valve in the crimped state, the initial size of the central opening 312 can be selected to be smaller than the working diameter of the guest valve. This will ensure that as the guest valve is expanded from the crimped state to the working diameter, the guest valve will contact the central lasso portion 304.

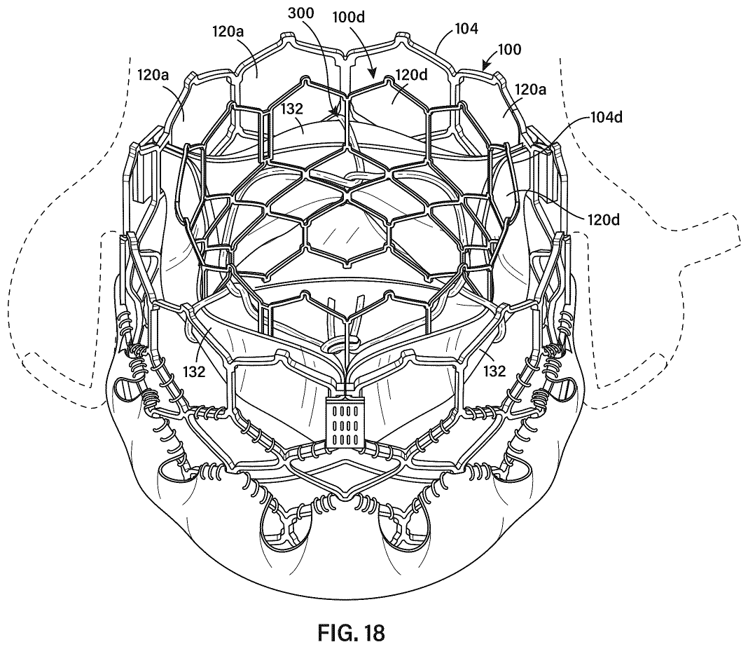

As the guest valve 100d is expanded, the guest valve 100d will come into contact with the central lasso portion 304 and exert radial force on the central lasso portion 304 that results in radial expansion of the central lasso portion 304 and folding of the host leaflets 132. FIG. 18 shows the guest valve 100d after it has been radially expanded to the working diameter, with the host leaflets 132 folded and pinned between the frame 104 of the host valve 100 and the frame 104d of the guest valve 100d. In the interest of clarity, some details of the guest valve 100d, such as a leaflet assembly, are not shown. However, these details can be similar to that of the prosthetic heart valve 100. Notably, as can be seen in FIG. 18, the outflow cells 120a of the host valve 100 remain substantially uncovered by the folded leaflets 132 and provide a pathway for the flow of blood. During diastole, blood can flow through the outflow cells 120d of the guest valve 100d and the outflow cells 120a of the host valve 100 to the coronary ostia.

One advantage of using the guest valve 100d as the expanding member is that a separate process of advancing an inflation tool to the host valve 100 to apply tension to the central lasso portion 304 of the leaflet lasso 300 can be avoided. In addition, the likelihood of the folded host leaflets 132 popping out from their folded positions prior to implanting the guest valve 100d can be reduced when the folding of the host leaflets 132 occurs simultaneously with expansion of the guest valve 100d.

Returning to FIG. 9, radiopaque markers 364 can be provided in the central lasso portion 304 to enable detection of the position of the central lasso portion 304 using X-ray/fluoroscopy imaging during installation of the leaflet lasso 300 on a leaflet assembly and/or during positioning of a guest valve within the opening defined by the central lasso portion 304. In some cases, the radiopaque markers 364 can be provided in one or more of the lateral lasso portions 308 to further enhance detection of the position of the leaflet lasso.

The radiopaque markers 364 can have any suitable shape (e.g., localized spots, lines, curves, and large areas on the lasso portions). The radiopaque markers can be incorporated into the leaflet lasso 300 using any suitable method. For example, radiopaque material could be incorporated into the tension member used to form the lasso portions 304, 308. In another example, radiopaque ink or other radiopaque coating can be applied on the desired portions of the leaflet lasso.

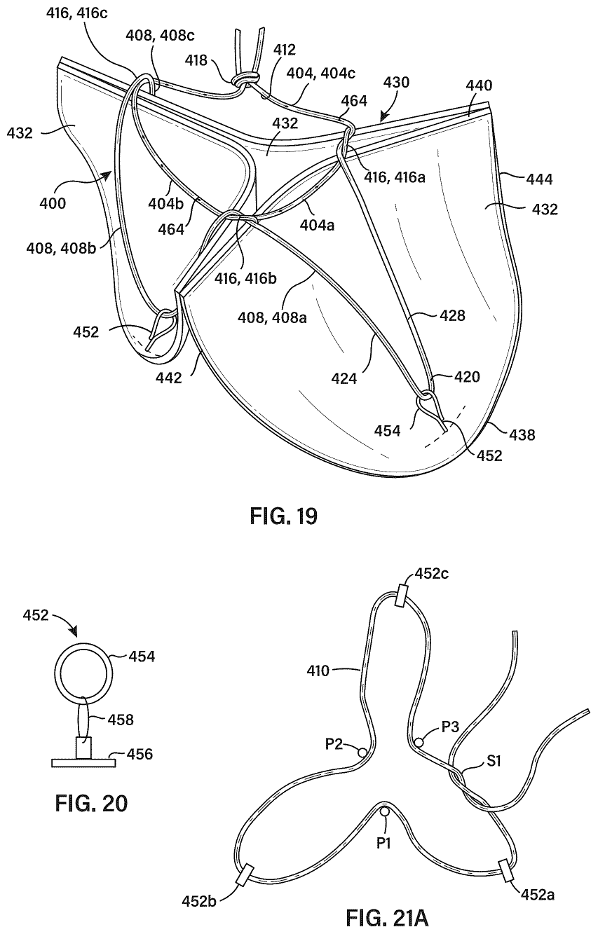

FIG. 19 illustrates an exemplary leaflet lasso 400 (also referred to herein as a leaflet lasso device or leaflet folding device), according to another implementation. For illustrative purposes, the leaflet lasso 400 is depicted as attached to a representative host leaflet assembly 430, which can be the leaflet assembly of a native heart valve or the leaflet assembly of an artificial valve implanted within a native heart valve.

The host leaflet assembly 430 includes one or more host leaflets 432 (three host leaflets are shown for illustrative purposes). Each of the host leaflets 432 can have a cusp edge 438, a free edge 440, and opposite side edges 442, 444 (as previously described with reference to FIG. 3). When the host leaflet assembly 424 is part of a heart valve, the free edges 440 move towards or away from each other to close or open the valve. The cusp edges 438 can be attached to a frame (in the case of a prosthetic heart valve) or to an annulus (in the case of a native heart valve). Although not shown, the side edges 442, 444 of adjacent host leaflets 432 can be attached together (e.g., by suturing). Although not shown, the side edges 442, 444 can have tabs (as illustrated, for example, in FIG. 3), which can be used to attach the leaflet assembly to a frame (in the case of a prosthetic heart valve).

The leaflet lasso 400 includes a central lasso portion 404 and one or more lateral lasso portions 408. In the illustrated example of FIG. 19, three lateral lasso portions 408 are shown, corresponding to the three host leaflets 432 of the example host leaflet assembly 424. In other examples, the number of lateral lasso portions 408 can be greater than or less than the number of leaflets in the host leaflet assembly to which the leaflet lasso is attached or is to be attached.

In the illustrated example, the central lasso portion 404 includes a first central lasso segment 404a extending across a lateral lasso portion 408a, a second central lasso segment 404b extending across a lateral lasso portion 408b, and a third central lasso segment 404c extending across a lateral lasso portion 408c. The central lasso segments 404a, 404b, 404c can be arranged to form a closed loop that defines a central opening 412. The central lasso portion 404 can have a loop length that is defined by the sum of the lengths of the central lasso segments 404a, 404b, 404c. The leaflet lasso 400 can be formed from a tension member having tail end portions fastened together by a locking knot 418 (e.g., a double knot or square knot). The locking knot 418 can be considered as part of the central lasso portion 404.

Each lateral lasso portion 408 can include strand portions 424, 428. The lateral lasso portion 408 can further include an anchor portion 420 extending between the strand portions 424, 428. The anchor portion 420 can have a U-shape or V-shape, for example. The strand portions 424, 428 can be connected at one end to the legs of the anchor portion 420 and coupled at another end to the central lasso portion 404 via slip knots 416 (or sliding knots). The slip knots 416 can be, for example, half knots (or single overhand knots). The slip knots 416 allow mutual adjustment of the length of the central lasso portion 404 and the lengths of the lateral lasso portions 408. The lateral lasso portion 408 can have a length that is defined by the sum of the lengths of the strand portions 424, 428 and the anchor portion 420.

In the illustrated example, the strand portions 424, 428 of the lateral lasso portion 408a are coupled to the central lasso portion 404 via spaced slip knots 416a, 416b, the strand portions of the lateral lasso portion 408b are coupled to the central lasso portion 404 via spaced slip knots 416b, 416c, and the strand portions of the lateral lasso portion 408c are coupled to the central lasso portion 404 via spaced slip knots 416c, 416a. As illustrated, adjacent lateral lasso portions have a common slip knot between them. For example, the slip knot 416a is common to lateral lasso portions 408a, 408c, the slip knot 416b is common to lateral lasso portions 408a, 408b, and the slip knot 416c is common to lateral lasso portions 408b, 408c.

The central lasso segment 404a extends between the slip knots 416a, 416b, the central lasso segment 404b extends between the slip knots 416b, 416c, and the central lasso segment 404c extends between the slip knots 416c, 416a. The central lasso segment 404c can include the locking knot 418 to close and secure the loop formed by the central lasso portion 404.

The leaflet lasso 400 can further include tissue anchors 452. FIG. 20 illustrates one example of a tissue anchor 452 including an eyelet member 454 coupled to an anchor member 456 (such as a T-bar anchor) by a loop 458. Alternatively, the eyelet 454 can be directly connected to the anchor member 456 without a loop 458. As shown in FIG. 19, each lateral lasso portion 408 can extend through the eyelet 454 of a respective tissue anchor 452 such that the tissue anchor 452 is slidably retained on the anchor portion 420 of the lateral lasso portion 408.

The tissue anchors 452 can be used to attach the leaflet lasso 400 to host leaflets. To attach the lateral lasso portions 408 to the host leaflets 432, the anchor member 456 (e.g., T-bar anchor shown in FIG. 20) of each tissue anchor 452 can be inserted through the tissue of a respective host leaflet 432, placing the anchor member 456 on an inner side of the host leaflet. The lateral lasso portion 408 will be on the outer side of the host leaflet 432, as shown in FIG. 19. In the anchored position of the leaflet lasso 400, the central lasso portion 404 and the slip knots 416 can extend over the free edges 440 of the host leaflets 432. Each slip knot 416 can extend over the coaptation region formed by the free edges 440 of two adjacent leaflets.

Tension can be applied to the central lasso portion 404 to radially expand the central lasso portion 404. As the central lasso portion 404 is radially expanded, the central lasso portion 404 will take up material from the strand portions 424, 428 of the lateral lasso portions 408 via the slip knots 416, reducing the length of the strand portions 424, 428 and axially contracting (or shortening) the lateral lasso portions 408. The lateral lasso portions 408 may contract by the same amount or by different amounts, depending on the uniformity of the expansion force acting along the central lasso portion 404. FIG. 19 shows each lateral lasso portion 408 extending along an axial direction of a respective host leaflet 432. In this case, contraction of a lateral lasso portion 408 anchored to a host leaflet 432 results in folding and/or displacement of the host leaflet 432 in the axial direction.

One advantage of the leaflet lasso 400 is that the leaflet lasso 400 can be constructed outside of the patient's body since the anchor portion 420 of the lateral lasso portion 408 does not need to be looped through the leaflet tissue. The leaflet lasso 400 can be delivered to an implantation site in the constructed state and attached to host leaflets at the implantation site using the tissue anchors 452. It can be faster and simpler to install the leaflet lasso 400 compared to the case where construction and attachment of the leaflet lasso to host leaflets at the implantation site occur together. Constructing the leaflet lasso outside of the patient's body can also mean better control of the construction of the lasso. In alternative examples, the leaflet lasso 400 can be pre-assembled on a prosthetic valve before it is implanted within a patient. In either case, the leaflet lasso can incorporate sufficient slack along the lateral lasso portions 408 to allow the host leaflets 432 to open and close in a normal manner under the flow of blood until a guest prosthetic valve is implanted within the host leaflets 432.

Any appropriate transcatheter instrument can be used to attach the tissue anchors 452 to the host leaflets at the implantation site. In one example where the tissue anchors 452 include T-bar anchor members 456 (shown in FIG. 20), a transcatheter delivery device having a mechanism similar to a commercially available tagging gun for attaching plastic barbs of hang tags to garments can be adapted for use within a patient's body to penetrate and extend the T-bar anchor members 456 through leaflet tissue material. Other examples of suture securing devices that may be used to attach tissue anchors to host leaflets are disclosed in U.S. Patent Application No. 63/211,254 (filed Jun. 16, 2021), the disclosure of which is incorporated herein by reference. In one example, the leaflet lasso can be delivered to the implantation site via the patient's vasculature, and the delivery apparatus can include the surgical instrument to attach the tissue anchors.

The central lasso portion 404 and the lateral lasso portions 408 of the leaflet lasso 400 can be constructed from a tension member that can be made from any of the various materials described above in connection with the leaflet lasso 400.

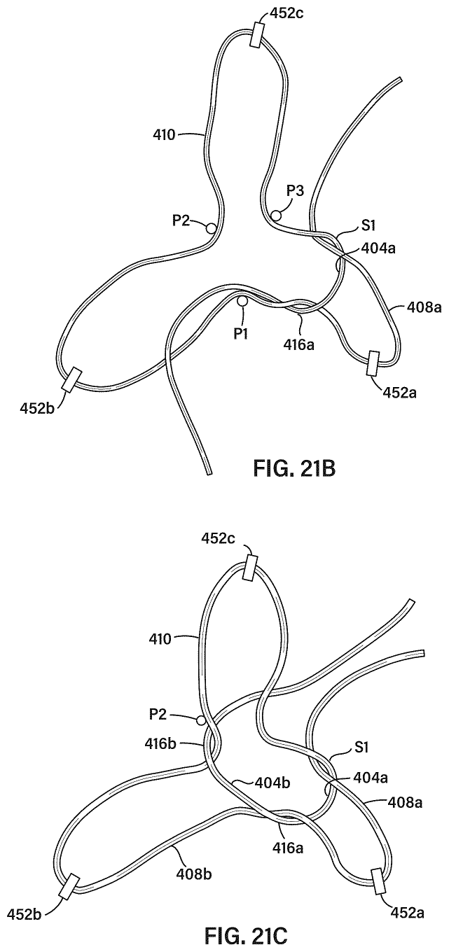

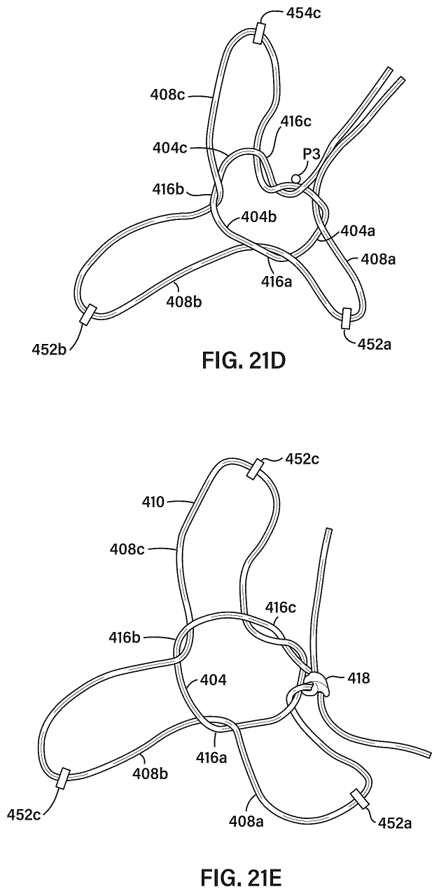

FIGS. 21A-21E illustrate one example of constructing the leaflet lasso 400 using a tension member 410 and the tissue anchors 452. The tension member 410 can be fed through the eyelet members of the tissue anchors 452a, 452b, 452c, as illustrated in FIG. 21A. A slip knot S1 can be formed to temporarily secure the free ends of the tension member 410. There are three points P1, P2, P3 where slip knots are to be formed along the tension member 410. As shown, the point P1 is between the tissue anchors 452a, 452b, the point P2 is between the tissue anchors 452b, 452c, and the point P3 is between the tissue anchors 452c, 452a. A first portion of the tension member between the points P3 and P1 will form a first lateral lasso portion, a second portion of the tension member between the points P1 and P2 will form a second lateral lasso portion, and a third portion of the tension member between the points P2 and P3 will form a third lateral lasso portion.

As illustrated in FIG. 21B, a first slip knot 416a is formed at point P1, completing a first lateral lasso portion 408a. A segment 404a of the central lasso portion extends between the temporary slip knot S1 and the first slip knot 416a. In FIG. 21C, a second slip knot 416b is formed at point P2, completing a second lateral lasso portion 408b. A segment 404b of the central lasso portion extends between the first slip knot 416a and the second slip knot 416b. In FIG. 21D, a third slip knot 416c is formed at point P3, completing a third lateral lasso portion 408c. A segment 404c of the central lasso portion extends between the second slip knot 416b and the third slip knot 416c. The central lasso portion 404 can be completed by securing the free ends of the tension member 410 with a locking knot 418, as shown in FIG. 21E. The initial size of the central lasso portion 404 as well as the initial sizes of the lateral lasso portions 408a, 408b, 408c can be adjusted via the slip knots 416a, 416b, 416c.

In an alternative implementation, the tissue anchors can be initially omitted from the leaflet lasso. The leaflet lasso without the tissue anchors can be delivered to the implantation site, accompanied by a suture securing device (or other surgical instrument) and the tissue anchors. After positioning each lateral lasso portion of the leaflet lasso at the desired host leaflet, the suture securing device can be operated to deploy a tissue anchor to secure the lateral lasso portion to the leaflet. For example, tissue anchors in the form of crimp members can be deployed to secure the lateral lasso portion to the leaflet.

Returning to FIG. 19, radiopaque markers 464 can be provided in the central lasso portion 404 to enable detection of the position of the central lasso portion 404 using X-ray/fluoroscopy imaging during installation of the leaflet lasso 400 on a leaflet assembly and/or during positioning of a guest valve within the opening defined by the central lasso portion 404. In some cases, the radiopaque markers 464 can be provided in one or more of the lateral lasso portions 408. The radiopaque markers 464 can have any suitable shape (e.g., localized spots, lines, curves, and large areas on the lasso portions) and can be incorporated into the leaflet lasso 400 using any suitable method, as previously described in connection with the leaflet lasso 400.

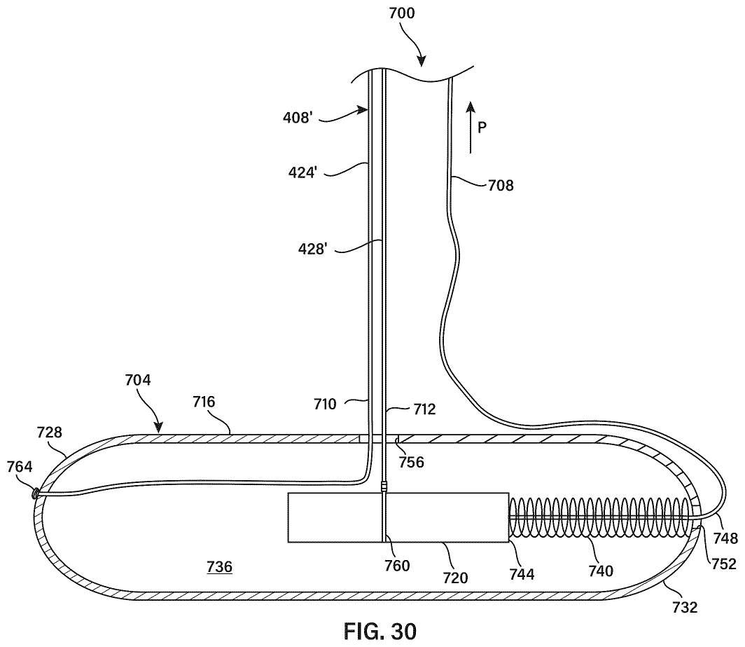

FIG. 30 illustrates an exemplary lasso anchor system 700 that can allow a leaflet lasso to be retrievable after the leaflet lasso has been used to fold host leaflets, according to another implementation. The lasso anchor system 700 includes a tissue anchor 704, strand portions of a lateral lasso portion of a leaflet lasso (e.g., strand portions 424′, 428′ of a lateral lasso portion 408′), and a release member 708. In the illustrated example, a U-shaped or V-shaped anchor portion (depicted as 420 in FIG. 19) does not extend between the strand portions 424′, 428′. Instead, the strand portions 424′, 428′ have tail end portions 710, 712 that can be used to couple the strand portions 424′, 428′ to the tissue anchor 704.

The tissue anchor 704 includes a shell member 716 and a core member 720. The shell member 716 is hollow and has closed end portions 728, 732. The shell member 716 can have an elongated shape. The closed end portions 728, 732 of the shell member 716 can be rounded (e.g., have a bull nose shape) to facilitate insertion of the shell member 716 through the tissue or body of a host leaflet and to avoid sharp edges in general. The core member 720 can have an elongated shape (e.g., a cylindrical shape). The core member 720 is disposed within a cavity 736 of the shell member 716 and can be substantially longitudinally aligned with the shell member 716.

The tissue anchor 704 can further include a resilient member (e.g., spring 740) disposed between an end 744 of the core member 720 and the end portion 732 of the shell member 716. The spring 740 can be attached to both the end 744 of the core member 720 and the end portion 732 of the shell member 716. In one example, the spring 740 can be a compression spring that normally resiliently biases the core member 720 in a direction away from the end portion 732. The spring 740 allows the core member 720 to be axially displaced relative to the shell member 716 under certain conditions (such as when a pull force is applied to the core member 720 that overcomes the force of the spring 740).

In one example, the release member 708 (e.g., a cable, suture, wire, or the like) is attached to the core member 720. For example, a tail end portion 748 of the release member 708 can extend from outside the tissue anchor 704, through an opening 752 in the end portion 732 of the shell member 716, into the cavity 736 defined within the shell member 716. The tail end portion 748 of the release cable 708 can further extend through the windings of the spring 740 to the end 744 of the core member 720 and can be attached to the end 744 of the core member 720. Thus, the release member 708 can be used to apply a pull force to the core member 720 that axially displaces the core member 720.

In one example, the spring constant of the spring 740 can be set to prevent spontaneous or inadvertent axial displacement of the core member 720 while allowing the core member 720 to compress the spring 740 when the release cable 708 applies a certain amount of pull force to the core member 720. The release member 708 can be attached to a tensioning system in a delivery apparatus configured to deliver a leaflet lasso and/or a guest valve to an implantation site.

In one example, the tail end portions 710, 712 of the strand portions 424′, 428′ can extend into the cavity 736 formed within the shell member 716 through an opening 756 formed in the wall of the shell member 716. In one example, the tail end portion 712 of the strand portion 428′ can include a loop 760 that can be extended around the circumference of the core member 720 such that the strand portion 428′ is coupled to the core member 720 by the loop 760. The strand portion 428′ can be detached from the core member 720 by slipping the loop 760 off the core member 720.

In one example, the tail end portion 710 of the strand portion 424′ is coupled to the shell member 716. For example, the tail end portion 710 can be retained on the end portion 728 by extending an end of the tail end portion 710 through an opening in the end portion 728 of the shell member 716 and forming a knot 764 at that end. The knot 764 can be secured to the shell member 716 by adhesive. In other examples, the tail end portion 710 can be retained on the end portion 728, or the body of the shell member 716, without a knot (e.g., by adhesive, welding, and the like).

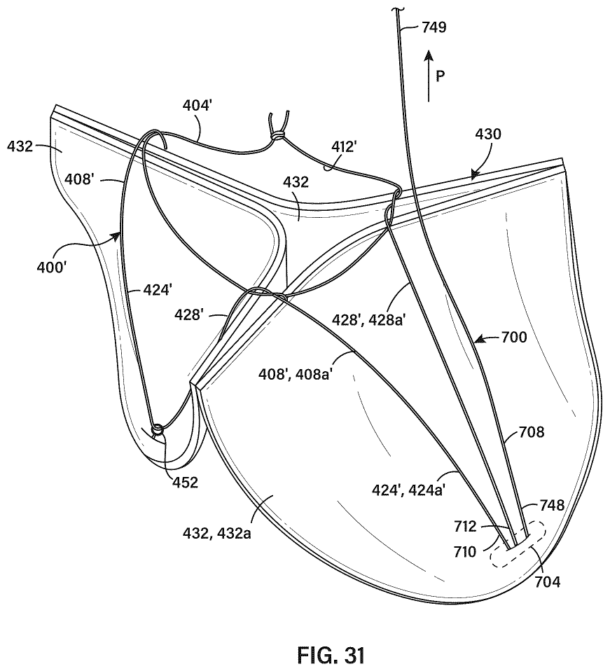

FIG. 31 shows the lasso anchor system 700 integrated with a leaflet lasso 400′. The leaflet lasso 400′ includes a central lasso portion 404′ and lateral lasso portions 408′. The central lasso portion defines a central opening 412′. In this example, the configuration of the central lasso portion 404′ and lateral lasso portion 408′ can be as described for the leaflet lasso 400 in FIG. 19. However, the lasso anchor system 700 is not limited to the particular leaflet lasso configuration shown in FIG. 19 and can be used with any leaflet lasso configuration described herein. The central lasso portion 404′ is shown positioned over the free edges of the host leaflets 432 of the host leaflet assembly 430 with each lateral lasso portion 408′ extending axially along a respective host leaflet 432.

In the example illustrated in FIG. 31, a lateral lasso portion 408′ (408a═) can be fastened to a respective host leaflet 432a by coupling the strand portions 424′ (424a═), 428′ (428a═) of the lateral lasso portion 408′ (408a═) to a tissue anchor 704 (as illustrated in FIG. 30) and inserting the tissue anchor 704 through the body of the host leaflet 432a (e.g., using a transcatheter delivery apparatus having a functionality similar to tagging gun or another suitable securing device). In the illustrated example, after insertion of the tissue anchor 704 through the body of the host leaflet 432a, the tissue anchor 704 is disposed on the inner side of the host leaflet 432a, while the strand portions 424a′, 428a′ are on the outer side of the host leaflet 432a. The tail end portions 710, 712 of the strand portions 424a′, 428a′ can be seen extending through the host leaflet 432a to the tissue anchor 704 disposed on the inner side of the host leaflet 432a.

While the strand portion 428′ is engaged with the tissue anchor 704 (as illustrated in FIG. 30), the elongated shape of the core member 720 and the tail end portion 712 of the strand portion 428′ looped around the core member 720 can serve to maintain the tissue anchor 704 in a substantially horizontal configuration (i.e., orthogonal to the axial axis of the host leaflet 432a and to the strand portions 424′, 428′). Also shown extending through the host leaflet 432a is the tail end portion 748 of the release member 708 that is attached to the shell member 716 of the tissue anchor 704. The end portion 749 of the release member 708 that is not attached to the tissue anchor 704 can be connected to a tensioning system of a delivery apparatus.

As previously described, the host leaflets 432 can be folded by applying tension to the central lasso portion 404′. The tension applied to the central lasso portion 404′ radially expands the central lasso portion 404′, resulting in axial contraction of the lateral lasso portions 408′ (due to the mutual adjustability of the central lasso portion 404′ and lateral lasso portions 408′) and folding of the host leaflets 432. As previously described, tension can be applied to the central lasso portion 404′ by positioning an expandable member in the central opening of the central lasso portion 404′ and expanding the expandable member against the central lasso portion 404′.

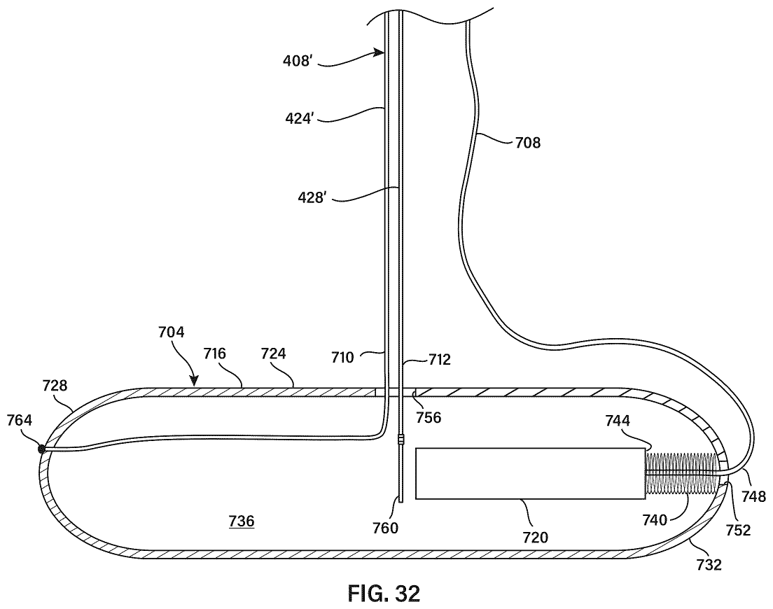

Referring to FIGS. 31-32, at the end of a leaflet folding procedure, the release member 708 can be pulled in a proximal direction P or tensioned such that the core member 720 is pulled against the spring 740. The pull force applied to the core member 720 results in compression of the spring 740 and axial displacement of the core member 720 relative to the shell member 716. As the core member 720 is displaced axially and against the spring 740, the core member 720 slips out of the loop 760 formed by the tail end portion 712 of the strand portion 428′, as illustrated in FIG. 32. The tail end portion 712 is thereby released from the core member 720.

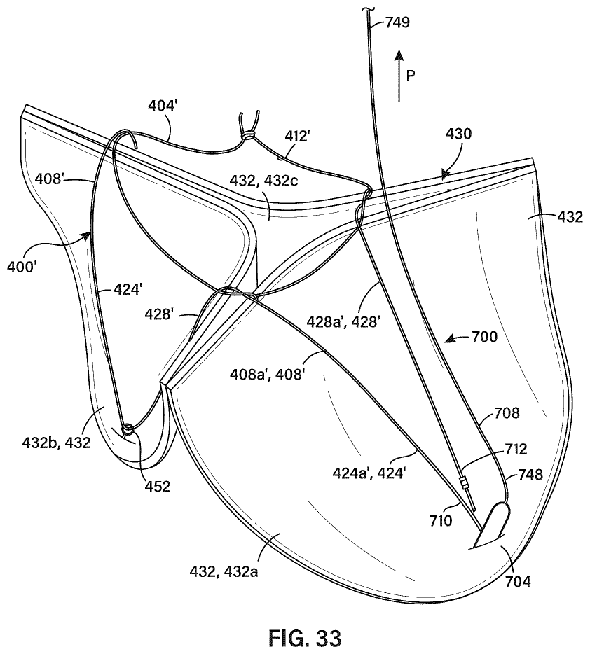

Further pulling of the core member 720 in a direction against the spring 740 can result in the free tail end portion 712 being pulled through the opening 756 in the wall of the hollow shell member 716, allowing at least a portion of the leaflet lasso 400′ to assume an elongated tensioned configuration. Additional pulling of the core member 720 by the release cable 708 can serve to rotate the tissue anchor 704 to an orientation that will allow the tissue anchor 704 to be pulled through the host leaflet 432 (as illustrated in FIG. 33) and retrieved, for example, into a shaft of a delivery apparatus. Since the leaflet lasso 400′ is attached to the tissue anchor 704 via the strand portion 424′ that is coupled to the shell member 716, the leaflet lasso 400′ will be retrieved with the tissue anchor 704.

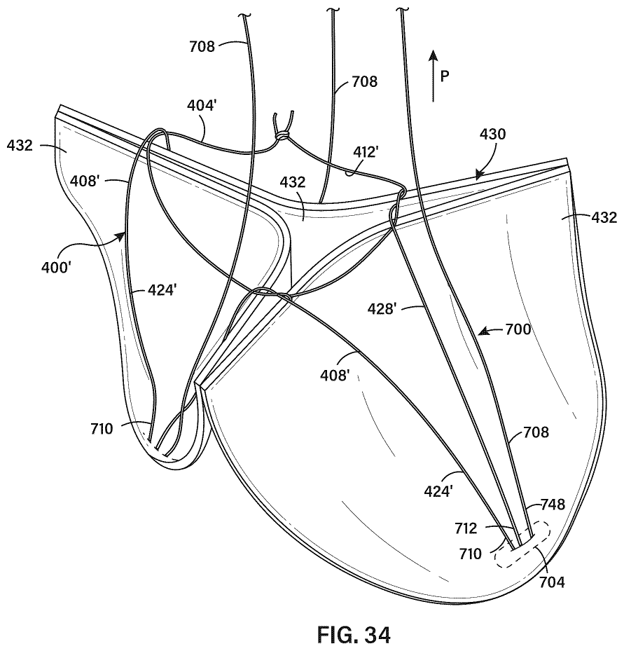

FIGS. 31 and 33 show the lasso anchor system 700 provided at one of a plurality of lateral lasso portions 408′ (e.g., lateral lasso portion 408a═). In one example, tissue anchors 452 can be provided at the other lateral lasso portions 408′ without release members. In this case, the release member 708 of the sole lasso anchor system 700 can be effective in pulling the tissue anchor 704 from the respective host leaflet 432a, and further pulling of the release member 708 can result in successive pulling of the other tissue anchors 452 at the other host leaflets 432b, 432c. Alternatively, as illustrated in FIG. 34, a lasso anchor system 700 can be provided at each of the plurality of lateral lasso portions 408′, each having a respective release member 708. The release members 708 of these lasso anchor systems can be coupled to a tensioning system of a delivery apparatus and work synergistically to release the leaflet lasso 400′ from the host leaflets and retrieve the leaflet lasso 400′.

A leaflet lasso 400′ can be constructed with a tissue anchor 704 attached to one or more lateral lasso portions 408′ of the leaflet lasso. The leaflet lasso 400′ can be delivered to an implantation site using a transcatheter delivery apparatus. In some cases, the transcatheter apparatus can be a delivery apparatus used to deliver a guest valve to an implantation site. At the implantation site, the central lasso portion 404′ of the leaflet lasso 400′ can be positioned over the free edges of the host leaflets 132, 432 at the implantation site (as illustrated for host leaflets 432 in FIG. 31). The host leaflets 132, 432 at the implantation site can be leaflets of a native valve or leaflets of a prosthetic valve implanted within a native valve. At least one of the lateral lasso portion 408′ can be fastened to a respective host leaflet 132, 432 at the implantation site by the tissue anchor 704 (as illustrated for host leaflet 432 in FIGS. 31 and 34). The tissue anchor 704 or other type of tissue anchor can be used for fastening the remaining lateral lasso portions to the remaining host leaflets.

The lasso anchor system 700 does not change the manner in which the leaflets are folded from what was previously described with reference to FIGS. 16-18. For example, the central lasso portion 404′ of the leaflet lasso 400′ can be tensioned to fold and/or axially displace the host leaflets (as previously described with reference to FIGS. 16-18). Tensioning of the central lasso portion 404′ can be accomplished using an expansion member such as an inflatable balloon or by positioning a guest valve in a central region of the host leaflet assembly and radially expanding the guest valve against the central lasso portion 404′ (as illustrated using guest valve 100d in FIG. 17). After the host leaflets 132, 432 have been folded, and in some cases after radially expanding the guest valve to a working diameter (or functional size), the release cable(s) 708 can be operated to detach the leaflet lasso 400′ from the host leaflet(s) 132, 432 and retrieve the leaflet lasso, as explained above.

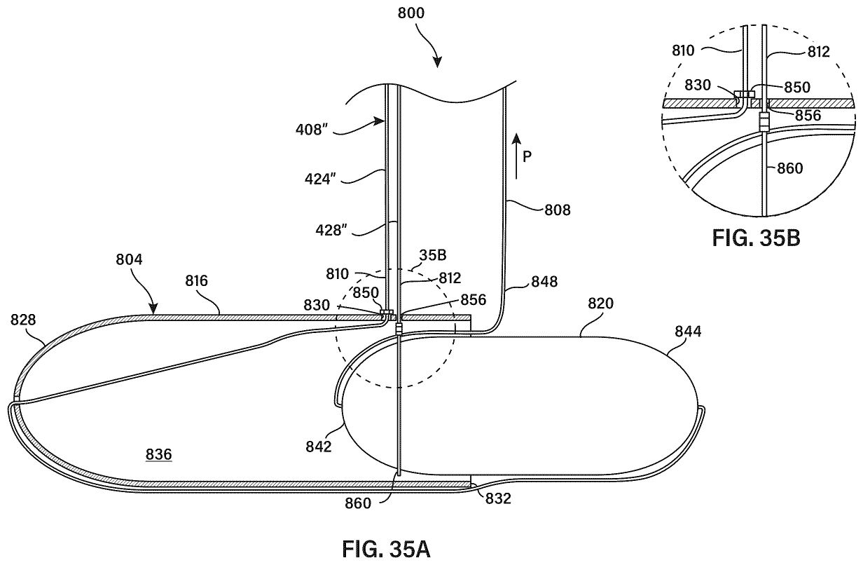

FIG. 35A illustrates a lasso anchor system 800 that can allow a leaflet lasso to be retrievable after a leaflet folding procedure using the leaflet lasso, according to another implementation. The lasso anchor system 800 includes a tissue anchor 804, strand portions of a lateral lasso portion of a leaflet lasso (e.g., strand portions 424″, 428″ of a lateral lasso portion 408″), and a release member 808. The strand portions 424″, 428″ are provided with separate tail ends 810, 812 (instead of being connected to the legs of a U-shaped or V-shaped anchor portion as depicted in FIG. 19). The tail ends 810, 812 can be used to couple the strand portions 424″, 428″ to the tissue anchor 804.

In the illustrated example, the tissue anchor 804 includes a shell member 816 and a core member 820. The shell member 816 is hollow and has a closed end portion 828 and an open end 832. The shell member 816 can have an elongated shape. The core member 820 is positioned within a cavity 836 of the shell member 816 such that an outer end portion 844 of the core member 820 protrudes from (or is exposed at) the open end 832 of the shell member 816 and an inner end portion 842 of the core member 820 is disposed within a cavity 836 of the shell member 816. The core member 820 can have an elongated shape (such as a capsule shape). The closed end portion 828 of the shell member 816 and the outer end portion 844 of the core member 820 can be rounded (e.g., have a bull nose shape) to facilitate insertion of the tissue anchor 804 through the tissue or body of a host leaflet and to avoid sharp edges in general.

In one example, the release member 808 (e.g., a cable, suture, wire, and the like) is attached to the core member 820. For example, a tail end 848 of the release member 808 can extend into the cavity 836 of the shell member 816 through the open end 832 of the shell member 816. The tail end 848 can be between the shell member 816 and the core member 820 and can be attached to the core member 820 (e.g., attached to the end portion 842 of the core member 820). In this way, the release member 808 can be used to apply a pull force to the core member 820 that axially displaces the core member 820 relative to the shell member 816. The release member 808 can be attached to a tensioning system in a delivery apparatus.

The tail ends 810, 812 of the strand portions 424″, 428″ extend into the cavity 836 of the shell member 816 through an opening 856 (as depicted in FIG. 35B) formed in the wall of the shell member 816. In one example, the tail end 812 can include a loop 860 that can be extended around the circumference of the core member 820 such that the strand portion 428″ is coupled to the core member 820 by the loop 860. The strand portion 428″ can be detached from the core member 820 by slipping the loop 860 off the core member 820.

In one example, the tail end 810 extends through the cavity 836 and out of an opening 830 formed in the end portion 828 of the shell member 816. The tail end 810 extends along the exterior of the shell member 816 and along the exterior of the portion of the core member 820 protruding from the open end 832 of the shell member 816. Finally, the tail end 810 is attached to the core member 820 (e.g., attached to the outer end portion 844 of the core member 820). The strand portion 428″ can include a stopper 850 (such as a knot or bead) formed at the entry point of the opening 830 (as depicted in FIG. 35B). The stopper 850 can engage the shell member 816 such that the length of the tail end 810 extending from the opening 830 into the cavity 836 and up to the attachment point at the core member 820 can be kept constant. The length of the tail end 810 extending from the opening 830 to the attachment point at the core member 820 can be set such that the core member 820 retains its position relative to the shell member 816 and cannot be spontaneously displaced.

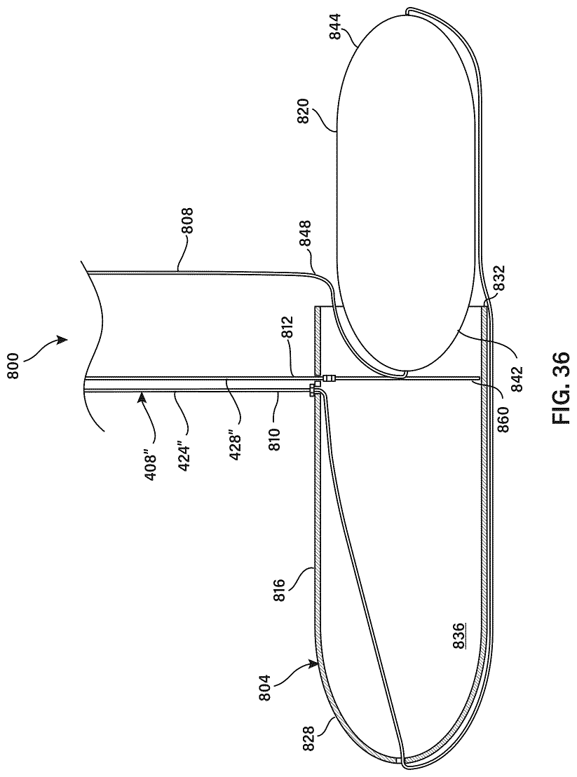

At the end of a leaflet folding procedure, the release member 808 can be pulled in a proximal direction P such that the core member 820 slides axially relative to the shell member 816 in a direction towards the open end 832 of the shell member 816. The length of the core member 820 can be set such that once the core member 820 is pulled axially, the loop 860 formed around the core member 820 by the tail end 812 slips off the core member 820 (as illustrated in FIG. 36). Rounding of the inner end portion 842 of the core member 820 can aid in slipping the loop 860 off the core member 820. Further pulling of the release member 808 can result in the tail end 812 being pulled through the opening 830 such that the leaflet lasso can assume an elongated tensioned configuration. Further pulling of the release member 808 can serve to reorient and serially pull the shell member 816 and core member 820 (since the shell member 816 and core member 820 are coupled together by the tail end 810). The shell member 816 and core member 820 can be retrieved into a shaft of a delivery apparatus along with the leaflet lasso that is attached to the shell member 816 via the tail end 810. The leaflet lasso can be removed from the patient's body with the delivery apparatus.

The lasso anchor system 800 can be incorporated into a leaflet lasso and used to attach the leaflet lasso to host leaflets in the same manner described for the lasso anchor system 700. Advantageously, both lasso anchor systems 700 and 800 allow the leaflet lasso and tissue anchors to be removed from the patient's body once the host leaflets are adequately folded to prevent or reduce the risk of coronary obstruction, thereby eliminating risks of long-term body reaction to the presence of such components. Although the lasso anchor systems 700, 800 have been described with respect to the leaflet lasso configuration depicted in FIG. 19, it should be understood that the lasso anchor systems 700, 800 are not limited to the leaflet lasso configuration depicted in FIG. 19 and can be used with any leaflet lasso configuration described herein, where the strand portions of the lateral lasso portions can be used as the datum for incorporating the lasso anchor systems.

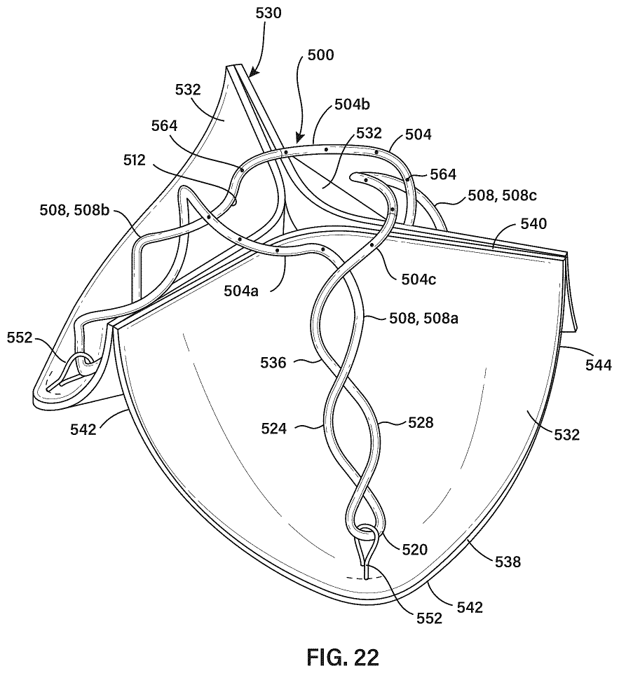

FIG. 22 illustrates an exemplary leaflet lasso 500 (also referred to herein as a leaflet lasso device or leaflet folding device), according to another implementation. For illustrative purposes, the leaflet lasso 500 is depicted as attached to a representative host leaflet assembly 530, which can be a leaflet assembly of a native heart valve or a leaflet assembly of an artificial valve implanted within a native heart valve.

The host leaflet assembly 530 includes one or more host leaflets 532 (three host leaflets are shown for illustrative purposes). Each of the host leaflets 532 can have a cusp edge 538, a free edge 540, and opposite side edges 542, 544 (as previously described with reference to FIG. 3). When the host leaflet assembly 530 is part of a heart valve, the free edges 540 move towards or away from the center of the valve to close or open the valve. The cusp edges 538 can be attached to a frame (in the case of a prosthetic heart valve) or to an annulus (in the case of a native heart valve). Although not shown, the side edges 542, 544 of adjacent host leaflets 532 can be attached together (e.g., by suturing). Although not shown, the side edges 542, 544 can have tabs (as illustrated, for example, in FIG. 3), which can be used to attach the leaflets to a frame (in the case of a prosthetic heart valve).

The leaflet lasso 500 includes a central lasso portion 504 and one or more lateral lasso portions 508. In the illustrated example of FIG. 22, three lateral lasso portions 508 (508a, 508b, 508c) are shown, corresponding to the three host leaflets 532 of the example host leaflet assembly 530. In other examples, the number of lateral lasso portions 508 can be greater than or less than the number of host leaflets in the host leaflet assembly to which the leaflet lasso is attached or is to be attached.

In the illustrated example, the central lasso portion 504 has a first central lasso segment 504a extending between the lateral lasso portions 508a and 508b, a second central lasso segment 504b extending between the lateral lasso portions 508b and 508c, and a third central lasso segment 504c extending between the lateral lasso portions 508c and 508a. The central lasso segments 504a, 504b, 504c can be arranged to form a central opening 512. The central lasso portion 504 can have a length that is defined by the sum of the lengths of the central lasso segments 504a, 504b, 504c.

Each lateral lasso portion 508 can include strand portions 524, 528. The lateral lasso portion 508 can further include an anchor portion 520 extending between the strand portions 524, 528. The anchor portion 520 can have a U-shape or V-shape, for example. The strand portions 524, 528 can be connected at one end to the legs of the anchor portion 520 and at another end to two different central lasso segments of the central lasso portion 504. For example, for the lateral lasso portion 508a, the strand portion 524 is connected to the central lasso segment 504a, and the strand portion 528 is connected to the central lasso segment 504c. The strand portions 524, 528 of each lateral lasso portion 508 are wound together to form an interwound portion 536 that extends between the central lasso portion 504 and the anchor portion 520. The lateral lasso portion 508 can have a length that is defined by an axial length of the interwound portion 536.

Since the strand portions 524, 528 of the lateral lasso portions 508 are connected to the segments of the central lasso portion 504, the length of the central lasso portion 504 and the lengths of the lateral lasso portions 508 are mutually adjustable. For example, increasing the length of the central lasso portion 504 will reduce the length of one or more of the lateral lasso portions 508 as the central lasso portion 504 takes up material from the lateral lasso portions 508. The length of the lateral lasso portions 508 is reduced by tightening the winding of the interwound portion 536 such that the axial length of the interwound portion 536 is shortened. As illustrated in FIG. 22, in the installed position of the leaflet lasso 500, the interwound portion 536 extends axially along the respective host leaflet 532. Thus, shortening of the interwound portion 536 results in folding and/or displacement of the leaflet along the axial direction of the leaflet.

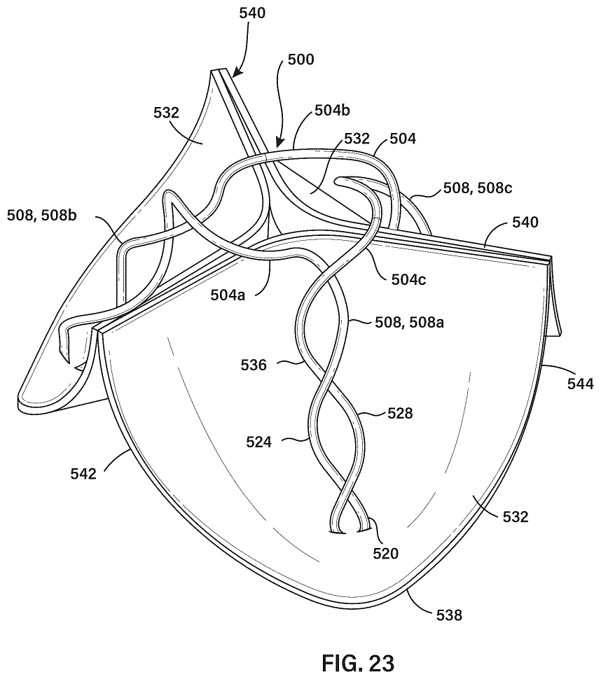

As illustrated in FIG. 22, each lateral lasso portion 508 can be fastened to a host leaflet 532 by a tissue anchor 552, which can allow the leaflet lasso 500 to be constructed outside of the patient's body, as previously described for the leaflet lasso 400. The tissue anchor 552 can be a T-bar anchor (such as depicted in FIG. 20) or a different type of tissue anchor. Alternatively, any of the previously described tissue anchor systems 700, 800 can be used to fasten the lateral lasso portion 508 to the host leaflet 532, as illustrated in FIGS. 30-36. Alternatively, each lateral lasso portion 508 could be looped through a host leaflet 532, as illustrated in FIG. 23. In this case, construction and attachment of the leaflet lasso can occur at an implantation site within the patient's body. In alternative examples, the leaflet lasso 500 can be pre-assembled on a prosthetic valve before it is implanted within a patient. In either case, the leaflet lasso can incorporate sufficient slack along the lateral portions 508 to allow the host leaflets 532 to open and close in a normal manner under the flow of blood until a guest prosthetic valve is implanted within the host leaflets 532.

The central lasso portion 504 and lateral lasso portions 508 of the leaflet lasso 500 can be constructed from a tension member comprising any of the various materials or configurations described above for leaflet lassos 300 and 400.

A tension member having a length sufficient to form the leaflet lasso 500 can be initially formed into a closed loop. Then, portions of the tension member spaced along the loop can be twisted to form the interwound portions 536 and the anchor portions 520 of the lateral lasso portions 508. The portions of the tension member between the interwound portions 536 will define the central lasso portion 504.

In one example, as illustrated in FIG. 22, the leaflet lasso 500 can include radiopaque markers 564 in the central lasso portion 504. The radiopaque markers 564 can enable detection of the position of the central lasso portion 504 using X-ray/fluoroscopy imaging during installation of the leaflet lasso 500 and during a valve implantation procedure. In some cases, radiopaque markers can be provided in one or more of the lateral lasso portions 508. The radiopaque markers 564 can have any suitable shape (e.g., localized spots, lines, curves, and large areas on the lasso portions). The radiopaque markers 564 can be incorporated into the leaflet lasso 500 using any suitable method, as previously described for leaflet lasso 300.

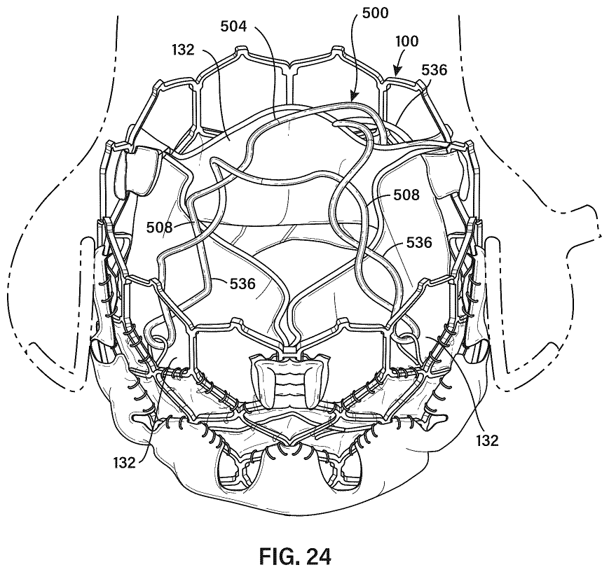

FIG. 24 shows the leaflet lasso 500 attached to the leaflets 132 of the prosthetic heart valve 100. Radial expansion of the central lasso portion 504 by an expansion member inserted through the opening of the central lasso portion 504 will shorten (or contract) the interwound portions 536 of the lateral lasso portions 508, resulting in axial folding and/or displacement of the leaflets 132. The expansion member can be, for example, an inflatable balloon or a guest valve to be installed within the prosthetic heart valve 100 in a ViV procedure.

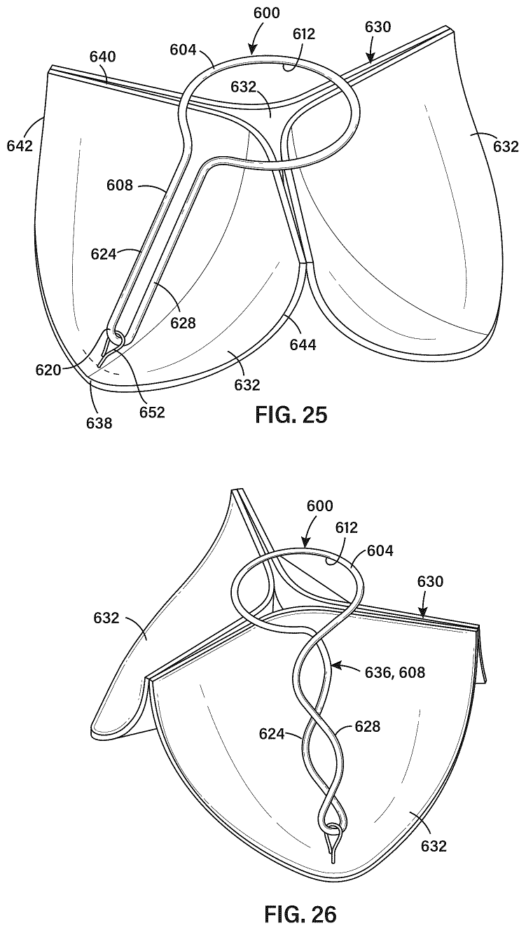

FIG. 25 illustrates an exemplary leaflet lasso 600 (also referred to herein as a leaflet lasso device or leaflet folding device), according to another implementation. For illustrative purposes, the leaflet lasso 600 is depicted as attached to a representative host leaflet assembly 630, which can be a leaflet assembly of a native heart valve or a leaflet assembly of an artificial valve implanted within a native heart valve.

The host leaflet assembly 630 includes one or more host leaflets 632 (three host leaflets are shown for illustrative purposes). Each of the host leaflets 632 can have a cusp edge 638, a free edge 640, and opposite side edges 642, 644 (as previously described with reference to FIG. 3). When the host leaflet assembly 630 is part of a heart valve, the free edges 640 move towards or away from the center of the valve to close or open the valve. The cusp edges 638 can be attached to a frame (in the case of a prosthetic heart valve) or to an annulus (in the case of a native heart valve). Although not shown, side edges 642, 644 of adjacent host leaflets 632 can be attached together (e.g., by suturing). Although not shown, the side edges 642, 644 can have tabs (as illustrated, for example, in FIG. 3), which can be used to attach the leaflets to a frame (in the case of a prosthetic heart valve).

The leaflet lasso 600 includes a central lasso portion 604 and a lateral lasso portion 608. The central lasso portion 604 can have a circular or elliptical shape for positioning over the free edges 640 of the leaflets 632. The central lasso portion 604 can define a central opening 612.

The lateral lasso portion 608 can include strand portions 624, 628. The lateral lasso portion 608 can further include an anchor portion 620 extending between the strand portions 624, 628. The strand portions 624, 628 can be connected at one end to the legs of the anchor portion 620 and at another end to the central lasso portion 604. The central lasso portion 604 and lateral lasso portion 608 together form a single loop in the example illustrated in FIG. 25. The length of the central lasso portion 604 and length of the lateral lasso portion 608 along the loop are mutually adjustable (e.g., increasing the length of the central lasso portion 604 will decrease the length of the lateral lasso portion 608).

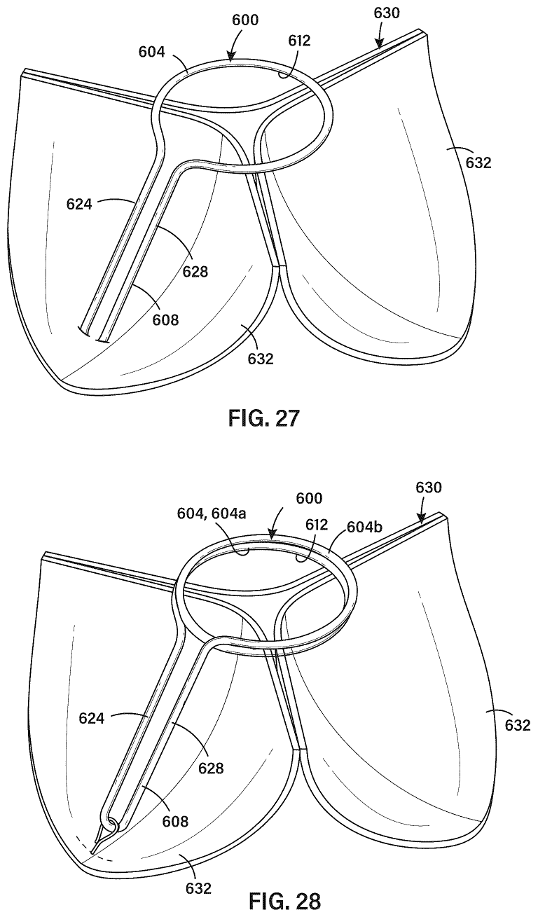

In one example, the strand portions 624, 628 can initially extend generally in parallel to each other (i.e., before applying tension to the central lasso portion 604). In another example, as illustrated in FIG. 26, the strand portions 624, 628 can be wound together to form an interwound portion 636.

As illustrated in FIG. 25, the lateral lasso portion 608 can be fastened to a host leaflet 632 by a tissue anchor 652. Alternatively, any of the previously described tissue anchor systems 700, 800 can be used to fasten the lateral lasso portion 608 to the host leaflet 632, as illustrated in FIGS. 30-36. Alternatively, the lateral lasso portion 608 can be looped through one of the host leaflets 632, where the anchor portion 620 of the lateral lasso portion 608 is disposed on the inner side of the host leaflet 632 and the strand portions 624, 628 are disposed on the outer side of the host leaflet 632, as illustrated in FIG. 27. While the lateral lasso portion 608 is attached to the host leaflet 632, tension applied to the central lasso portion 604 to expand the central lasso portion 604 will be effective in contracting the lateral lasso portion 608 and folding the host leaflet 632.

In FIG. 25, the central lasso portion 604 forms a partial loop that does not span an entire circular perimeter of 360 degrees. As such, the degree to which the lateral lasso portion can be contracted may be somewhat lessened compared to the previous examples of leaflet lassos. The degree to which the lateral lasso portion 608 can be contracted can be increased by increasing the number of windings/loops in the central lasso portion 604 such that each additional winding/loop advantageously serves to amplify the shortening (or contraction) of the lateral lasso portion 608.

FIG. 28 shows an example where the central loop portion 604 is wound to form two loops 604a, 604b defining a central opening 612. The extreme end portions of the two loops 604a, 604b are connected to the lateral lasso portion 608 such that the lengths of the loops 604a, 604b and the length of the lateral lasso portion 608 are mutually adjustable. When tension is applied to the central loop portion 604 (e.g., via an expansion member extending through the central opening 612), each of the loops included in the central loop portion 604 has an opportunity to radially expand by taking up material from the lateral lasso portion 608. Since each loop of the central loop portion 604 is taking up material from the lateral lasso portion 608, each additional central loop serves to amplify contraction of the lateral lasso portion 608, resulting in greater folding of the leaflet to which the lateral lasso portion is attached.

Any of the previously described leaflet lassos (300, 400, 500) can incorporate multiple windings in the central lasso portion in the same manner described for the leaflet lasso 600 above, where the multiple windings will serve to amplify contraction of the lateral leaflet lassos.

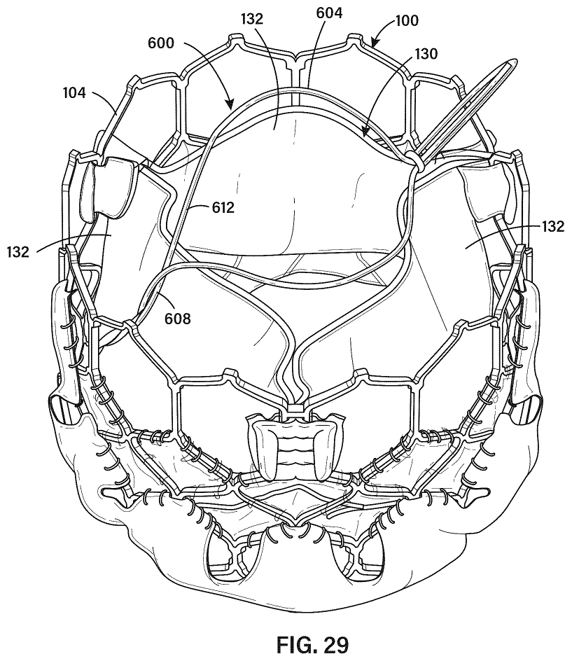

FIG. 29 shows the leaflet lasso 600 attached to the leaflet assembly 130 of the prosthetic heart valve 100. In particular, the central lasso portion 604 is disposed over the free edges of the leaflets 132 of the prosthetic heart valve 100, and the lateral lasso portion 608 is fastened to one of the leaflets 132. Radial expansion of the central lasso portion 604 by an expansion member inserted through the opening 612 formed by the central lasso portion 604 will axially shorten (or contract) the lateral lasso portion 608, resulting in axial folding of the leaflets 132. The expansion member can be, for example, an inflatable balloon or a guest valve to be installed within the prosthetic heart valve 100 in a ViV procedure.

The leaflet lasso 600 can be made from a tension member (e.g., suture, cable, or wire) as in the previous examples. When the leaflet lasso 600 is in the installed position, the central lasso portion 604 is on a plane that is at an angle relative to the plane of the lateral lasso portion 608 (e.g., the central lasso portion 604 and the lateral lasso portion 608 are on different planes that are neither parallel nor perpendicular to each other). The orientation of these planes can be such that the central lasso portion 604 can be positioned over the end of a leaflet assembly while the lateral lasso portion extends axially along a leaflet of the leaflet assembly.

For the case where the leaflet lasso 600 is fastened to a single leaflet, the stability of the leaflet lasso in the installed position can be improved by forming the central and lateral lasso portions (or at least the central lasso portion) from a relatively stiff material (such as a wire). Alternatively, the central and/or lateral lasso portions can comprise a relatively stiff layer (e.g., a relatively stiff outer sheath layer or relatively still inner core layer) that increases the stiffness of those portions of the leaflet lasso 600. The relatively stiff material can help the central lasso portion 604 to retain its position over the end of the leaflet assembly as the lateral lasso extends along one of the host leaflets. The relatively stiff material can also help the central lasso portion 604 retain a circular or elliptical shape, which would allow easier insertion of a prosthetic heart valve into the central opening 612 of the central lasso portion 604. Similarly, any of the leaflet lassos 300, 400, 500 can incorporate a relatively stiff material in the central lasso portion to help retain the circular or elliptical shape of the central lasso portion.

The leaflet lasso 600 can produce asymmetric folding of the leaflet assembly. For example, the folding can occur mainly at the single leaflet to which the leaflet lasso is attached. The asymmetric folding can be used when folding of a single leaflet will suffice to prevent coronary artery obstruction (e.g., if only one of the coronary ostia is at risk of obstruction and folding the leaflet close to that coronary ostium would suffice). Advantageously, a leaflet lasso that is attached to a single leaflet may be easier to assemble and use.

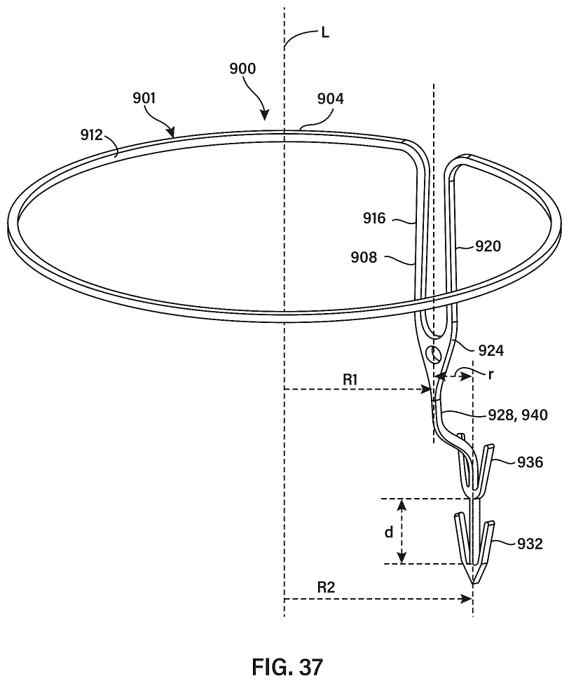

FIG. 37 illustrates an exemplary implantable leaflet folding device 900 (which can also be referred to as a leaflet lasso device or a leaflet lasso), according to another implementation. The leaflet folding device 900 can be attached to a host leaflet assembly, which can be a leaflet assembly of a native heart valve or a leaflet assembly of an artificial valve implanted within a native heart valve. The leaflet folding device 900 can be used to fold one or more leaflets of the host leaflet assembly (e.g., to reduce the risk of coronary artery obstruction after installation of a guest valve within the host leaflet assembly).

The leaflet folding device 900 includes a lasso member 901 having a central lasso portion 904 and a lateral lasso portion 908. The central lasso portion 904 can have a generally circular or elliptical shape for positioning over the free edges of the leaflets of a host leaflet assembly. The central lasso portion 904 can define a central opening 912. The central lasso portion 904 (or central opening 912) can have a central axis L defining an axial direction.

The lateral lasso portion 908 can be on a plane that is transverse to the plane of the central lasso portion 904. The lateral lasso portion 908 can include vertical members 916, 920 connected to end portions of the central lasso portion 904. The vertical members 916, 920 can be spaced apart in a direction along a perimeter of the central opening 912. The lateral lasso portion 908 can further include a web 924 extending between and connected to the end portions of the vertical members 916, 920. The lateral lasso portion 908 can be positioned at a radius R1 from the central axis L.

The central lasso portion 904 and the lateral lasso portion 908 together form a single loop (or continuous loop). In some examples, the circumferential length of the central lasso portion 904 and the axial length of the lateral lasso portion 908 are mutually adjustable along the loop (e.g., increasing the circumferential length of the central lasso portion 904 by radially expanding the central lasso portion 904 will take up material from the lateral lasso portion 908 and decrease the axial length of the lateral lasso portion 908). This mutual adjustment can be used to fold host leaflets.

The leaflet folding device 900 can include an anchor member 928 connected to the lasso member 901 at the web 924. The anchor portion 928 can include one or more anchors, such as a first anchor 932 (or distal anchor) and a second anchor 936 (or proximal anchor), which can be spaced from each other in the axial direction by a distance d. In one example, the distance d can be equal to or greater than a thickness of a base of the host leaflet to which the leaflet folding device 900 is to be anchored such that the thickness of the base of the host leaflet at the anchor location can be retained between the anchors 932, 936.

The first and second anchors 932, 936 can be positioned at a radius R2 from the central axis L. In one example, the radius R2 can be greater than the radius R1 such that the first and second anchors 932, 936 are radially offset from the lateral lasso portion 908 by a radial offset r (i.e., the anchors 932, 936 are radially outward of the lateral lasso portion 908). In one example, the radial offset r can be provided by a bent portion 940 (e.g., an elbow) extending between the web 924 and the second anchor 936.

In one example, the anchors 932, 936 can be V-shaped anchors. In one example, the first anchor 932 (or distal anchor) can have an atraumatic tip. In another example, the first anchor 932 can have a sharp tip that can be used to penetrate a host leaflet during installation of the leaflet folding device 900 on a host leaflet assembly. The anchors 932, 936 desirably are shaped to be pushed through a leaflet in one direction, but prevent or resist movement of the anchor in the opposite direction once it is pushed through the leaflet. In some examples, the prongs or legs of the anchors can be flexible and can deform or be pushed inwardly toward each other to facilitate insertion of an anchor through a leaflet.

In one example, the leaflet folding device 900 can be formed from a flexible member, such as a wire. In one example, the leaflet folding device 900 can be formed from a single piece of material (e.g., by laser cutting) such that the leaflet folding device 900 is seamless. In another example, the lasso member 901 and anchor member 928 can be formed separately and then attached together (e.g., by welding). The leaflet folding device 900 can be made from any biocompatible metal or alloy, e.g., stainless steel, titanium, and a nickel-titanium alloy (e.g., Nitinol). In one example, the leaflet folding device 900, or at least the lasso member 901, can be made from a shape memory material (e.g., Nitinol) to allow the leaflet folding device 900 to be foldable for delivery to an implantation location and then to unfold to a preconfigured, expanded shape.

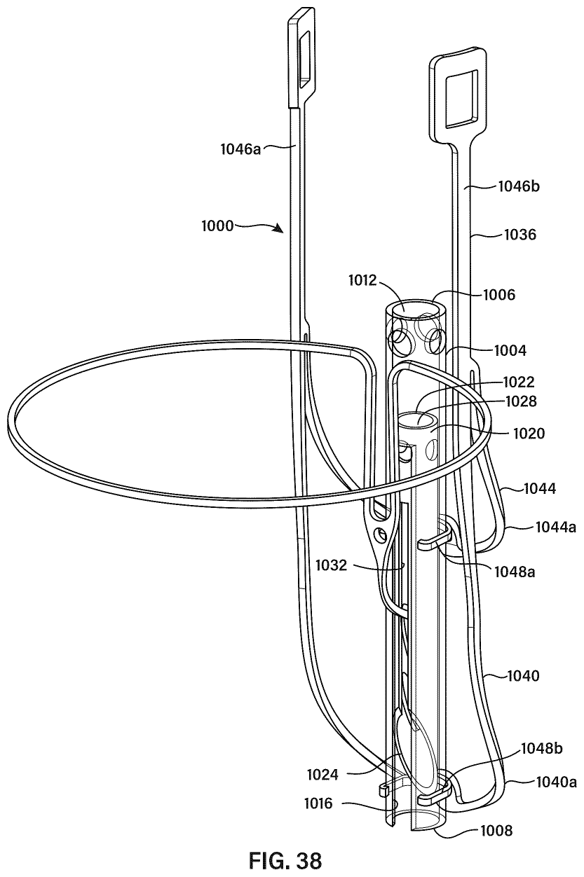

FIG. 38 illustrates a delivery accessory 1000 that can be used to deliver the leaflet folding device 900 and other leaflet folding devices described herein to an implantation location within a patient's body. The delivery accessory 1000 can be coupled to shafts or other components of a delivery apparatus and operated by a handle of the delivery apparatus.

The delivery accessory 1000 can include a tube 1004 having a proximal end 1006 and a distal end 1008. The tube 1004 has a lumen 1012 that extends from the proximal end 1006 to the distal end 1008. The tube 1004 can include a longitudinal slot 1016 extending proximally from the distal end 1008. The longitudinal slot 1016 extends through a wall thickness of the tube 1004 to connect to the lumen 1012.

The delivery accessory 1000 can include a hollow needle 1020 received within the lumen 1012. The hollow needle 1020 is longitudinally aligned with the tube 1004 and is movable relative to the tube 1004 along a longitudinal axis of the tube 1004. The hollow needle 1020 has a proximal end 1022 and a distal end 1024. The distal end 1024 can be beveled (or otherwise shaped) to form a sharp tip. The hollow needle 1020 has a lumen 1028 extending from the proximal end 1022 to the distal end 1024. The hollow needle 1020 can include a longitudinal slot 1032 extending proximally from the distal end 1024. The longitudinal slot 1032 extends through a wall thickness of the hollow needle 120 to connect to the lumen 1028.

The hollow needle 1020 can be shorter in length compared to the tube 1004 such that the hollow needle 1020 can be fully retracted within the tube 1004 for delivery to an implantation location. The longitudinal slot 1016 on the tube 1004 can be radially aligned with the longitudinal slot 1032 on the hollow needle 1020 to define an access passage to the lumen 1028 of the hollow needle 1020. In some cases, the hollow needle 1020 can include a guide element (such as a radially extending pin) that can be disposed in the longitudinal slot 1016 of the tube 1004 to radially align the longitudinal slots 1016, 1032.

The delivery accessory 1000 can in some examples include a stabilization member 1036 retained on the tube 1004. The stabilization member 1036 can help support the tube 1004 relative to a host leaflet during a leaflet anchoring procedure using the delivery accessory 1000. In one example, the stabilization member 1036 can include stabilization arms 1040, 1044 axially spaced apart from each other (i.e., spaced in a direction parallel to the longitudinal axis of the tube 1004) and extending laterally outward from the tube 1004. The stabilization arm 1044 has upper ends connected to respective leg portions 1046a, 1046b. Distal portions of the leg portions 1046a, 1046b extend distally beyond the stabilization arm 1044 and form the stabilization arm 1040. The stabilization member 1036 can include one or more attachment mechanisms for connecting the tube 1004 to the stabilization member 1036. For example, clipping members 1048a and 1048b can be connected to the stabilization arms 1040 and 1044, respectively. Each clipping member 1048a, 1048b can have opposing retaining arms that extend at least partially around opposing sides of the tube 1004 and retain the tube between the retaining arms via a snap-fit connection or a friction fit connection.

In use, the lower curved portion 1040a of the stabilization arm 1040 can extend into and engage the cusp behind a host leaflet 132a and the lower curved portion 1044a of the stabilization arm 1044 can engage the arterial wall (e.g., within the aortic root) at a location facing the exterior of the host leaflet 132a or downstream the host leaflet 132a such that the tube 1004 is supported between the host leaflet 132a and the arterial wall. This can facilitate insertion of the anchor portion of a leaflet folding device through the cusp or base of the host leaflet 132a.

In one example, the leaflet folding device 900 can be releasably coupled to the delivery accessory 1000 for delivery to an implantation location. For example, the leaflet folding device 900 can be coupled to the delivery accessory 1000 by inserting the anchor portion 928 of the leaflet folding device 900 through the passage formed by the radially aligned longitudinal slots 1016, 1032 to position the anchors 932, 936 within the lumen 1028 of the hollow needle 1020 and such that the bent portion 940 extends through the passage. The bent portion 940 can position the lasso member 901 (including the central lasso portion 904, the lateral lasso portion 908, and the web 924) outside of the tube 1004.

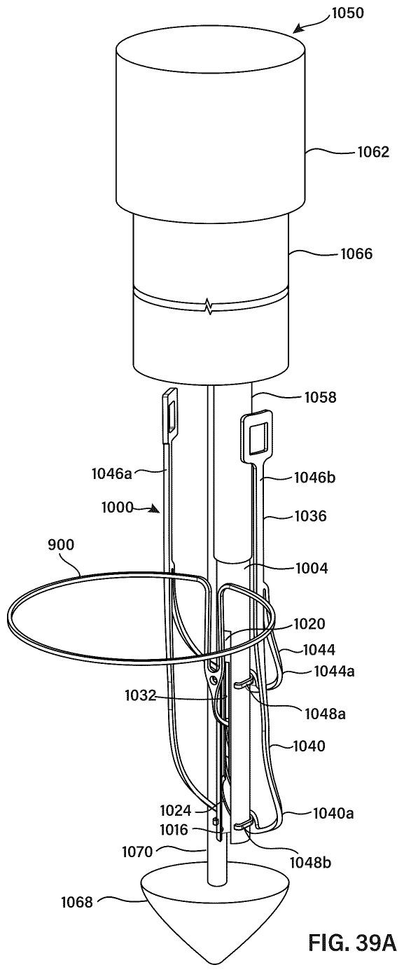

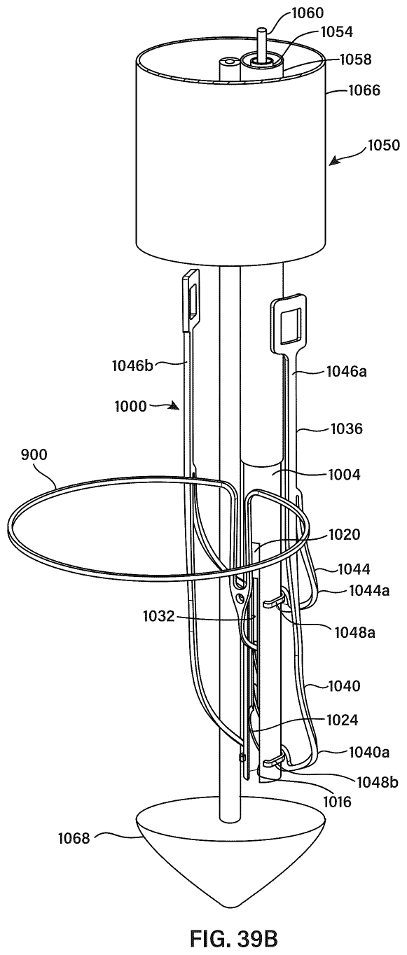

FIGS. 39A and 39B show the delivery accessory 1000 coupled to a delivery apparatus 1050, according to one example. For example, the hollow needle 1020 can be coupled to a first shaft 1054 of the delivery apparatus 1050. The tube 1004 can be coupled to a second shaft 1058 of the delivery apparatus 1050. The second shaft 1058 can extend over the first shaft 1054. The delivery apparatus 1050 can further include a pusher member 1060, such as in the form of a shaft extending coaxially through the first shaft 1054 and the needle 1020, for deploying the leaflet folding device from the needle 1020. The proximal end portions of the first and second shafts 1054, 1058 and the pusher member 1060 can be coupled to a handle 1062 of the delivery apparatus 1050, which can include respective mechanisms (e.g., knobs) for independently and axially displacing the shafts 1054, 1058 and the pusher member 1060 relative to each other and the handle body. In some examples, the delivery accessory 1000 can be removable from the delivery apparatus 1050. For example, the tube 1004 and the needle 1020 can be releasably coupled to their respective shafts 1058, 1054 of the delivery apparatus. In other examples, the delivery accessory 1000 can be an integral and non-removable component of the delivery apparatus. For example, the tube 1004 and needle 1020 can be extensions of shafts 1058 and 1054, respectively.

In one example, instead of coupling the second shaft 1058 to the tube 1004, or in addition to coupling the second shaft 1058 to the tube 1004, a respective shaft can be coupled to leg portions 1046a, 1046b of the stabilization member 1036. The proximal end of the shaft can be coupled to the handle 1062 so that the shaft can be displaced via a mechanism (e.g., a knob) of the handle 1062. In this example, displacement of the shaft in the proximal or distal direction produces corresponding displacement of the stabilization member 1036 in the same direction. Since the stabilization member 1036 is attached to the tube 1004, displacement of the shaft likewise results in displacement of the tube 1004 in the same direction. Therefore, a second shaft 1058 coupled to the tube 1004 and/or a shaft coupled to the stabilization member 1036 (when the delivery accessory 1000 includes the stabilization member 1036) can be used to displace the tube 1004. For example, the tube 1004 can be displaced to expose the hollow needle 1020 or to enclose the hollow needle 1020.

The delivery apparatus 1050 can further include an outer shaft 1066 that extends over the second shaft 1058. In one example, the outer shaft 1066 can be axially displaced in the distal direction, via the handle 1062. For example, the outer shaft 1066 can be axially displaced to enclose the delivery accessory 1000 and an attached leaflet folding device 900 within the lumen of the outer shaft 1066. In this manner, the distal end portion of the outer shaft 1066 functions as a sheath or capsule for containing the delivery accessory and the leaflet folding device during delivery through a patient's vasculature. The leaflet folding device, and in particular the lasso portion, can be compressed and/or folded to fit within the outer shaft for delivery. For example, the central lasso portion 904 can be folded distally relative to the lateral lasso portion 916 as well as pinched or flattened radially such that central lasso portion 904 deforms from a ring shape to an elongated or straight shape. The central lasso portion 904 can be placed in the folded and flattened state by advancing the outer shaft 1066 distally relative to the leaflet folding device 900 and the delivery accessory 1000 and/or retracting the leaflet folding device 900 and the delivery accessory 1000 into the outer shaft 1066. The central lasso portion 904, or the entire leaflet folding device 900, can be made from a shape memory material so that the central lasso portion 904 can self-expand from the folded and flattened state to its expanded, function shape (as shown in FIG. 37) when deployed from the outer shaft 1066, as further described below.

The delivery apparatus 1050 can include a nosecone 1068 attached to a distal end of a nosecone shaft 1070. The nosecone 1068 and nosecone shaft 1070 can define a central passage to receive a guidewire. The nosecone shaft 1070 can be axially displaceable via the handle 1062. The outer shaft 1066 can be advanced distally to contact the nosecone 1068 to enclose the delivery accessory 1000 and the attached leaflet folding device 900 retain within the shaft 1066. In this case, the nosecone 1068 will serve as a cover for the distal end of the lumen of the outer shaft 1066 when the delivery accessory 1000 and the leaflet folding device 900 are enclosed therein.

A method of delivering the leaflet folding device 900 can include coupling the leaflet folding device 900 to the delivery accessory 1000 (as depicted in FIG. 38), coupling the delivery accessory 1000 to the shafts of the delivery apparatus 1050 (as depicted in the FIGS. 39A and 39B) (if the delivery accessory is a separable component from the delivery apparatus), and loading the leaflet folding device and the delivery accessory into the outer shaft 1066. The distal end portion of the delivery apparatus 1050 can be advanced through a patient's vasculature to an implantation location. For example, if implanting the leaflet folding device on a host valve at the aortic valve position, the delivery apparatus can be advanced through a femoral artery and the aorta to the host valve in a retrograde delivery approach. The delivery apparatus can be advanced over a guidewire, which can be inserted into the passage defined by the nosecone 1068 and nosecone shaft 1070.

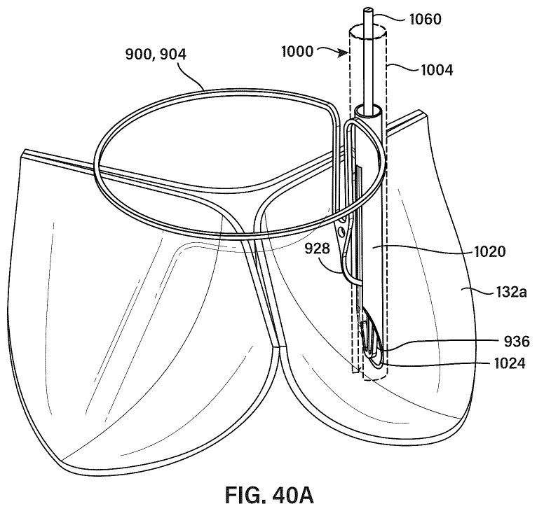

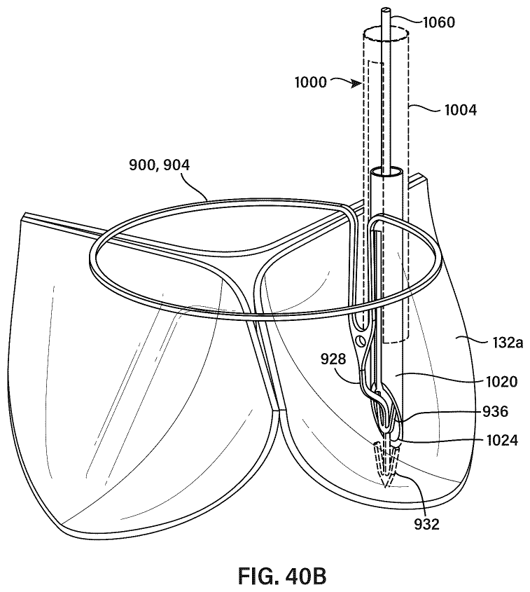

At the implantation location, the outer shaft 1066 can be retracted to expose the delivery accessory 1000 and the attached leaflet folding device 900. If formed from a shape memory material, the central lasso portion 904 can self-expand to its functional shape and size, as shown in FIG. 40A. The nosecone shaft 1070 can be advanced distally relative to the outer shaft 1066 to allow the nosecone 1068 to pass through a central portion of the host leaflet assembly. The tube 1004 can be positioned on a proximal side of the base of a host leaflet 132, as shown in FIG. 40A. The stabilization arms 1040, 1044 are not shown in FIGS. 40A and 40B. However, in another example where the delivery accessory 1000 includes the stabilization arms 1040, 1044, the lower stabilization arm 1040 can extend into the cusp behind the host leaflet 132a, and the upper stabilization arm 1040 can contact the arterial wall downstream of the cusp such that the tube 1004 is supported between the host leaflet 132a and the arterial wall.

After positioning the tube 1004 on the proximal side of the base of the host leaflet 132a, the hollow needle 1020 can be advanced distally to contact the proximal side of the base of the host leaflet 132. The tube 1004 can be displaced away from the proximal side of the base of the host leaflet 132 and in a proximal direction relative to the needle 1020 to expose the distal end portion of the hollow needle 1020, as shown in FIG. 40B. The hollow needle 1020 can be further advanced distally such that the distal end 1040 (or tip) of the hollow needle 1020 penetrates the base of the host leaflet 132a, as shown in FIG. 40B. After the distal end 1040 of the hollow needle 1020 penetrates the base of the host leaflet, the first anchor 932 can be advanced distally through the opening at the distal end 1024 of the hollow needle 1020 to the distal side of the base of the host leaflet, as shown in FIG. 40B. The first anchor 932 can be advanced from the needle 1020 by moving the pusher member 1060 distally. The pusher member abuts the anchor portion 928 and pushes the anchor portion 928 distally to deploy the first anchor 932 from the needle 1020 and push the first anchor through the leaflet.

The second anchor 936 is positioned at the proximal side of the base of the host leaflet so that a portion of the base of the host leaflet is retained between the first and second anchors 932, 936, thereby anchoring the leaflet folding device 900 to the host leaflet 132a. The hollow needle 1020 can be withdrawn from the host leaflet 132 after the first anchor 932 has been advanced through the hollow needle 1020 to the distal side of the base of the host leaflet 132a. The hollow needle 1020 can be retracted into the tube 1004 either by displacing the hollow needle 1020 proximally into the tube 1004 or extending the tube 1004 distally over the hollow needle 1020.

In some examples, the first anchor 932 can have a sharp tip to penetrate the base of the host leaflet 132a. In these examples, the hollow needle 1020 may or may not penetrate through the base of the host leaflet 132a. For example, the hollow needle 1020 can be positioned on the proximal side of the base of the host leaflet 132a. Then, the first anchor 932 can be advanced to penetrate the base of the host leaflet 132a and be disposed on the distal side of the base of the host leaflet 132a. In other examples, the hollow needle 1020 can be omitted entirely from the delivery accessory 1000 and the anchor portion 928 of the leaflet folding device 900 can be retained within the tube 1004 during delivery to an implantation site.

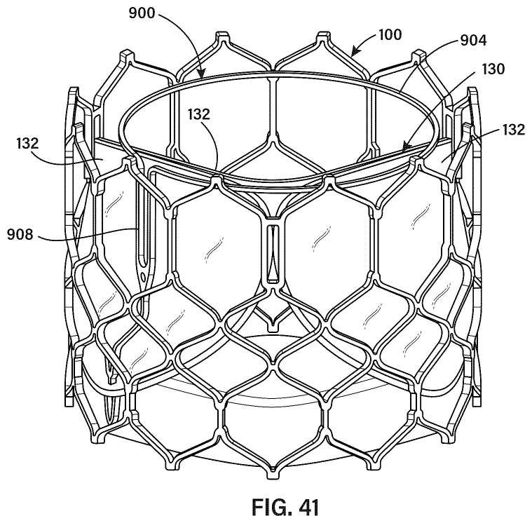

FIG. 41 shows the leaflet folding device 900 attached to the leaflet assembly 130 of the prosthetic heart valve 100. In particular, the central lasso portion 904 of the leaflet folding device 900 is disposed over the free edges of the leaflets 132 of the prosthetic heart valve, and the lateral lasso portion 908 of the leaflet folding device 900 is fastened to one of the leaflets 132. Radial expansion of the central lasso portion 904 by an expansion member inserted through the opening formed by the central lasso portion 904 will axially shorten (or contract) the lateral lasso portion 908, resulting in axial folding of the leaflets 132. The expansion member can be, for example, an inflatable balloon or a guest valve to be installed within the prosthetic heart valve 100 in a ViV procedure.

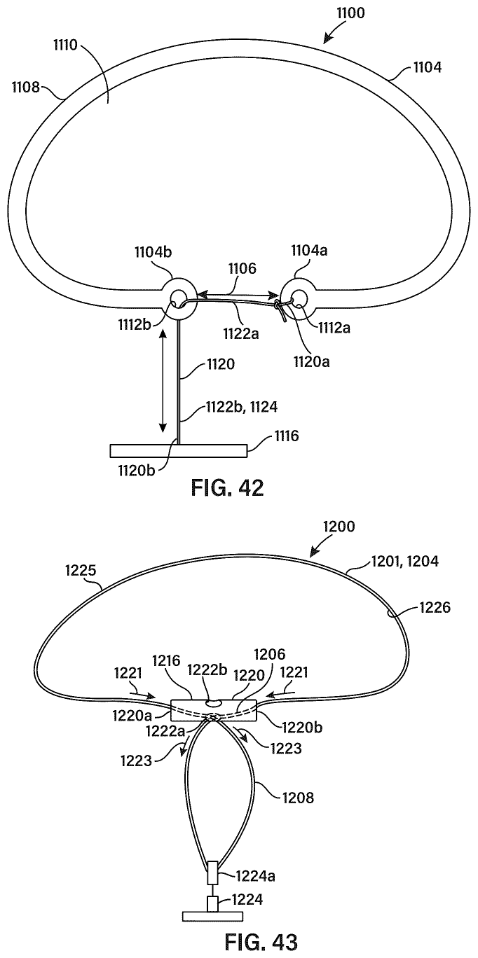

FIG. 42 illustrates an exemplary leaflet folding device 1100 (also referred to as a leaflet folding tool or leaflet lasso device), according to another implementation. The leaflet folding device 1100 can be attached to a host leaflet assembly, which can be a leaflet assembly of a native heart valve or a leaflet assembly of an artificial valve implanted within a native heart valve. The leaflet folding device 1100 can be used to fold one or more leaflets of the host leaflet assembly (e.g., to reduce the risk of coronary artery obstruction after installation of a guest valve within the host leaflet assembly).

The leaflet folding device 1100 includes a curved member 1104, an anchor member 1116, and a flexible tether 1120 (e.g., a suture) coupling the curved member 1104 to the anchor member 1116. The curved member 1104 has first and second end portions 1104a, 1104b, which are separated by a gap 1106. In the illustrated example, a first end portion 1120a of the flexible tether 1120 is attached to the first end portion 1104a. For example, the first end portion 1120a can be attached to an eyelet 1112a formed in the first end portion 1104a (or otherwise retained on the first end portion 1104a), such as by tying the first end portion 1120a to the eyelet 1112a. The flexible tether 1120 extends through an eyelet 1112b in the second end portion 1104b and is thereby slidable relative to the second end portion 1104b of the curved member 1104. A second end portion 1120b of the flexible suture 1120 is attached to the anchor member 1116.

A first portion 1122a of the flexible tether 1120 extends between the first end portion 1104a and the second end portion 1104b of the curved member 1104. A second portion 1122b of the flexible tether 1120 extends between the second end portion 1104b and the anchor member 1116. The lengths of the first portion 1120a and the second portion 1120b can change as the flexible tether 1120 slides through the eyelet 1112b. The first portion 1122a of the flexible tether 1120 and the curved member 1104 form a central lasso portion 1108 (or central loop) of a lasso member. The central lasso portion 1108 defines a central opening 1110. The second portion 1122b of the flexible tether 1120 forms a lateral lasso portion 1124 of the lasso member.

The central lasso portion 1108 can be positioned over the free edges of the leaflets of a host leaflet assembly. The central opening 1110 will allow access to a central portion of the host leaflet assembly while the central lasso portion 1108 is positioned over the host leaflet assembly. The length of the central lasso portion 1108 and the length of the lateral lasso portion 1124 are mutually adjustable. For example, when the central lasso portion 1108 is radially expanded, the central lasso portion 1108 will take up material from the lateral lasso portion 1124 (i.e., take material from the second portion 1122b of the flexible tether 1120 to increase the length of the first portion 1122a of the flexible tether 1120) such that the length of the central lasso portion 1108 is increased and the length of the lateral lasso portion 1124 is decreased.

The leaflet folding device 1100 can be delivered to an implantation site and fastened to a host leaflet using the anchor member 1116. The leaflet folding device 1100 can be delivered using the delivery accessory 1000. In one example, the leaflet folding device 1100 can be coupled to the delivery accessory 1000 by inserting the second portion 1122b of the flexible tether 1120 and the anchor member 1116 into the lumen of the hollow needle 1020. In another example, if the curved member 1104 of the leaflet folding device 1100 is flexible and made of a shape memory material, the leaflet folding device 1100 can be coupled to the delivery accessory by disposing the entire leaflet folding device 1100, in a folded configuration, within the lumen of the hollow needle 1020. In both examples, the anchor member 1116 can be positioned proximate the distal end 1024 (or tip) of the hollow needle 1020.

At the implantation location, the hollow needle 1020 can be positioned on a proximal side of the base of the host leaflet. The hollow needle 1020 can be advanced to penetrate the base of the host leaflet. After the hollow needle 1020 has penetrated the base of the host leaflet, the anchor member 1116 can be advanced through the distal end 1024 of the hollow needle 1020 to position the anchor member 1116 (or just a distal anchor of anchor member 1116) at a distal side of the base of the host leaflet. Thereafter, the hollow needle 1020 can be withdrawn from the host leaflet, leaving the anchor member 1116 in position to anchor the leaflet folding device 1100 to the host leaflet. If the entire leaflet folding device 1100 was initially disposed within the hollow needle 1020, withdrawal of the hollow needle 1020 can allow the curved member 1104 of the leaflet folding device 1100 to self-expand over the free edges of the host leaflets.

Once the leaflet folding device 1100 is anchored in place, the host leaflets are free to move between open and closed states. When the host leaflet to which the leaflet folding device 1100 is anchored transitions to the open state, the host leaflet pushes the second portion 1122b of the flexible tether 1120 (i.e., the lateral lasso portion 1124) of the leaflet folding device 1100 radially outward, which can advantageously serve to align the central lasso portion 1108 (i.e., the curved member 1104 and the first portion 1122a of the flexible tether 1120) of the leaflet folding device 1100 over the host leaflets.

In some examples, the anchor member 1116 can have the same or similar configuration as the anchor portion 928 of the leaflet folding device 900. Thus, the anchor member 1116 can have one or more anchors 932, 936, as depicted in FIG. 37. In this manner, the leaflet folding device 1100 is similar to the leaflet folding device 900, except that the lateral lasso portion 908 is replaced with a tether 1120.

The method of using a flexible tether to fasten a leaflet folding device to a host leaflet can be extended to other implementations herein (e.g., replacing slip knots and lateral lasso portions with spaced end portions in the central lasso portion that are slidably coupled together by a flexible suture, as described for the leaflet folding device 1100).

FIG. 43 illustrates an exemplary leaflet folding device 1200 (also referred to as a leaflet folding tool or leaflet lasso device), according to another implementation. The leaflet folding device 1200 can be attached to a host leaflet assembly, which can be a leaflet assembly of a native heart valve or a leaflet assembly of an artificial valve implanted within a native heart valve. The leaflet folding device 1200 can be used to fold one or more leaflets of the host leaflet assembly (e.g., to reduce the risk of coronary artery obstruction after installation of a guest valve within the host leaflet assembly).

The leaflet folding device 1200 includes a lasso member 1201 having a central lasso portion 1204 (or central loop) and a lateral lasso portion 1208 (or lateral loop). The leaflet folding device 1200 can include an anchor member 1224 (e.g., a T-bar anchor or an anchor member similar to anchor portion 928), which can be coupled to, or otherwise retained on, the lateral lasso portion 1208. The anchor member 1224 can be used to fasten the leaflet folding device 1200 to a host leaflet.

In the illustrated example, a portion 1206 of the lasso member 1201 connecting the central lasso portion 1204 to the lateral lasso portion 1208 is disposed within a guide element 1216 and is slidable relative to the guide element 1216. The lengths of the central lasso portion 1204 and the lateral lasso portion 1208 can be mutually adjusted through the guide element 1216 (e.g., when the central lasso portion 1204 is radially expanded, material can be pulled from the lateral lasso portion 1208 through the guide element 1216 such that the length of the central lasso portion 1204 is increased while the length of the lateral lasso portion 1208 is decreased).

In one example, the guide element 1216 can include a tube 1220 having one or more wall openings (e.g., wall openings 1222a, 1222b). To form the lasso member 1201, the ends of a continuous wire 1225 can be inserted into the lumen of the tube 1220 from openings at the opposite ends 1220a, 1220b of the tube 1220 to form the central lasso portion 1204, as indicated by arrows 1221. The ends of the continuous wire can then be passed out of the lumen of the tube 1220 through one of the wall openings (e.g., wall opening 1222a), as indicated by arrows 1223, to form the lateral lasso portion 1208. The continuous wire 1225 is free to slide through the openings at the ends 1220a, 1220b of the tube 1220 and through the wall opening 1222a, allowing the lengths of the central lasso portion 1204 and the lateral lasso portion 1208 to be mutually adjustable.

The ends of the continuous wire 1225 forming part of the lateral lasso portion 1208 can be coupled to the anchor member 1224 using any suitable method. For example, one end of the continuous wire 1225 can be threaded through an eyelet 1224a of the anchor member 1224 and then secured to the other end of the continuous wire.

The continuous wire 1225 can be made of NiTi (e.g., Nitinol) or other biocompatible metal or alloy. Forming the central lasso portion 1204 and lateral lasso portion 1208 from a continuous wire can simplify the construction of the leaflet folding device.

The central lasso portion 1204 can form a generally circular or elliptical shape defining a central opening 1226. When the leaflet folding device 1200 is implanted on a host leaflet assembly, the central lasso portion 1204 can be positioned over the free edges of the leaflets of the host leaflet assembly, where the central opening 1226 formed by the central lasso portion 1204 will allow access to the central portion of the host leaflet assembly. When the central lasso portion 1204 is positioned over the free edges of the host leaflets, the lateral lasso portion 1208 will extend axially along one of the host leaflets. The anchor member 1224 can be used to fasten the lateral lasso portion 1204 to the host leaflet.

The leaflet folding device 1200 can be coupled to the hollow needle 1020 of the delivery accessory 1000 such that the anchor member 1224 is disposed proximate to the distal end 1024 (or tip) of the hollow needle 1020. At the implantation site, the hollow needle 1020 can be extended into the base of a host leaflet. Then, the anchor member 1224 can be advanced through the distal end 1024 of the hollow needle 1020 to anchor the leaflet folding device 1200 in place, as described for the other implementations.

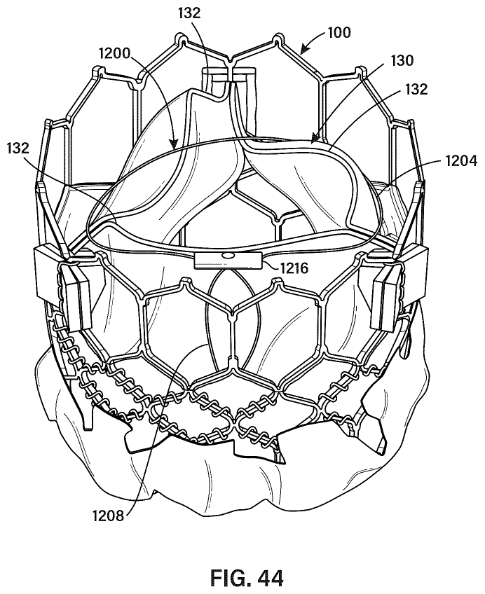

FIG. 44 shows the leaflet folding device 1200 attached to the leaflet assembly 130 of the prosthetic heart valve 100. In particular, the central lasso portion 1204 of the leaflet folding device 1200 is disposed over the free edges of the leaflets 132 of the prosthetic heart valve, and the lateral lasso portion 1208 of the leaflet folding device 1200 is fastened to one of the leaflets 132. Radial expansion of the central lasso portion 1204 by an expansion member inserted through the opening formed by the central lasso portion 1204 will axially shorten (or contract) the lateral lasso portion 1208, resulting in axial folding of the leaflets 132. The expansion member can be, for example, an inflatable balloon or a guest valve to be installed within the prosthetic heart valve 100 in a ViV procedure.

The various leaflet lasso (or leaflet folding device) examples (300, 400, 500, 600, 900, 1100, 1200) serve to fold and retain the host leaflets in the folded position during expansion of a guest valve from a crimped state to a working diameter. In cases where the host valve includes a frame, after the guest valve has been expanded to its working diameter, the leaflet lasso may no longer be needed. For example, the host leaflets may be retained effectively in the folded configuration by virtue of being constrained between the frames of the guest valve and host valve. In this case, the tension and/or tissue anchors used in constructing the various examples of the leaflet lassos can be made of bioresorbable material. This can advantageously reduce or eliminate risks of additional implantable components remaining within the patient's body after valve implantation and can simplify regulatory requirements. Alternatively, or in addition, the leaflet lassos can incorporate any of the releasable lasso anchor systems 700, 800, which can allow the leaflet lassos to be retrieved after the leaflet lassos are no longer needed.

A method of valve implantation using any of the leaflet lassos/leaflet folding devices (300, 400, 500, 600, 900, 1100, 1200) described herein can generally include installing a leaflet lasso on a host leaflet assembly. The host leaflet assembly can be a leaflet assembly of a native heart valve (such as a native aortic valve) or a leaflet assembly of an artificial valve implanted within a native heart valve. The installation can include positioning a central lasso portion of the leaflet lasso over an end of the host leaflet assembly (e.g., the end including the free edges of the leaflets of the host leaflet assembly) and attaching one or more lateral lasso portions of the leaflet lasso to one or more of the host leaflets.

The method can further include applying tension to the central lasso portion to radially expand the central lasso portion and axially contract (or shorten) the lateral lasso portion(s) attached to the host leaflet(s), resulting in folding of one or more of the host leaflets. Tension can be applied to the central lasso portion using an expansion member that is disposed within a central opening of the central lasso portion and expanded against the central lasso portion.

In one example, the method can further include delivering a prosthetic heart valve as a guest valve to the host valve. The prosthetic heart valve can be delivered in a crimped state. The prosthetic heart valve can be positioned within a central region formed between the host leaflet assembly. The method can include radially expanding the guest valve from the crimped state to a working diameter.

In some cases, applying tension to the central lasso portion and radially expanding the guest valve can be combined. The guest valve can be positioned to extend through both the central opening formed by the central lasso portion and the central region formed between the host leaflet assembly. As the guest valve is radially expanded, the guest valve can contact the central lasso portion and apply tension to the central lasso portion that results in folding of the one or more of the host leaflets.

In some cases, prior to installing the leaflet lasso, the leaflet lasso is constructed outside of the patient's body. The constructed leaflet lasso is then delivered to the implantation site (e.g., via a transcatheter apparatus) for installation on the host leaflet assembly. In one example the installation can include attaching tissue anchors of the leaflet lasso to one or more of the leaflets of the host leaflet assembly.

In some cases, installation of the leaflet lasso can include constructing the leaflet lasso at the implantation site. In some cases, one or more lateral loop portions of the leaflet lasso can be looped through one or more leaflets of the host leaflet assembly as the leaflet lasso is constructed.

In some cases, the leaflet lasso is constructed and attached to a prosthetic valve before it is implanted within a patient. The leaflet lasso can remain on the prosthetic valve for its useful life and is actuated to fold the leaflets when a new prosthetic valve is implanted in a ViV procedure.

In some cases, the leaflet lasso/leaflet folding device can be attached to a delivery accessory that can be coupled to a delivery apparatus for delivery to the implantation site. At the implantation site, the delivery accessory can be operated to deploy and attach the leaflet folding device to the host leaflet assembly.

Any of the leaflet folding devices, delivery apparatus, prosthetic valves, and medical assemblies disclosed herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Examples

In view of the implementations of the disclosed subject matter described above, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1: A leaflet lasso for modifying a leaflet of a host heart valve comprises a central lasso portion defining a central opening; and at least one lateral lasso portion coupled to the central lasso portion such that radial expansion of the central lasso portion by applying tension to the central lasso portion results in axial contraction of the lateral lasso portion.

Example 2: A leaflet lasso according to any example herein, particularly example 1, wherein the central lasso portion comprises a first loop, wherein the lateral lasso portion comprises a second loop, and wherein the first loop is coupled to the second loop by a slip knot.

Example 3: A leaflet lasso according to any example herein, particularly any of examples 1 to 2, wherein the central lasso portion and the lateral lasso portion are formed from a tension member having two extreme end portions fastened together by a lock knot, and wherein the central lasso portion comprises the lock knot.

Example 4: A leaflet lasso according to any example herein, particularly example 1, wherein the lateral lasso portion is coupled to the central lasso portion by two spaced slip knots, and wherein a segment of the central lasso portion extends between the two spaced slip knots.

Example 5: A leaflet lasso according to any example herein, particularly example 1, wherein the central lasso portion and the lateral lasso portion are on different planes and form a single loop.

Example 6: A leaflet lasso according to any example herein, particularly example 5, wherein the lateral lasso portion comprises two tie-in portions extending in an axial direction and an anchor portion, wherein first ends of the two tie-in portions are connected to the anchor portion, and wherein second ends of the two tie-in portions are connected to the central lasso portion.

Example 7: A leaflet lasso according to any example herein, particularly example 5, wherein the lateral lasso portion comprises two tie-in portions and an anchor portion, wherein the two tie-in portions are wound together to form an interwound portion, wherein a first end of the interwound portion is connected to the anchor portion, and wherein a second end of the interwound portion is connected to the central lasso portion.

Example 8: A leaflet lasso according to any example herein, particularly example 1, wherein the central lasso portion comprises a plurality of loops arranged to define the central opening.

Example 9: A leaflet lasso according to any example herein, particularly any of examples 1 to 8, further comprising a tissue anchor retained on the lateral lasso portion.

Example 10: A leaflet lasso according to any example herein, particularly example 9, wherein the tissue anchor comprises an eyelet member coupled to an anchor member, and wherein the lateral lasso portion extends through the eyelet member.

Example 11: A leaflet lasso according to any example herein, particularly example 10, wherein the anchor member is a T-bar anchor.

Example 12: A leaflet lasso according to any example herein, particularly any of examples 10-11, wherein the tissue anchor is bioresorbable.

Example 13: A leaflet lasso according to any example herein, particularly any of examples 1 to 12, wherein the central lasso portion and the lateral lasso portion are bioresorbable.

Example 14: A leaflet lasso according to any example herein, particularly any of examples 1 to 13, wherein the central lasso portion comprises one or more radiopaque markers.

Example 15: A leaflet lasso according to any example herein, particularly any of examples 1-14, wherein the at least one lateral lasso portion comprises a plurality of lateral lasso portions coupled to the central lasso portion.

Example 16: A leaflet lasso for modifying a leaflet of a host heart valve comprising a central lasso portion defining a central opening; and a plurality of lateral lasso portions, each of the lateral lasso portions coupled to the central lasso portion such that radial expansion of the central lasso portion by applying tension to the central lasso portion results in axial contraction of the lateral lasso portion.

Example 17: A leaflet lasso according to any example herein, particularly example 16, wherein the central lasso portion comprises a first loop, wherein each of the lateral lasso portions comprises a second loop, and wherein each of the second loop is coupled to the first loop by a slip knot.

Example 18: A leaflet lasso according to any example herein, particularly example 16, wherein each of the lateral lasso portions is coupled to the central lasso portion by two spaced slip knots, and wherein a segment of the central lasso portion extends between the two spaced slip knots.

Example 19: A leaflet lasso according to any example herein, particularly example 16, wherein each lateral lasso portion comprises two tie-in portions and an anchor portion, wherein the two tie-in portions are wound together to form an interwound portion, wherein a first end of the interwound portion is connected to the anchor portion, and wherein a second end of the interwound portion is connected to the central lasso portion.

Example 20: A leaflet lasso according to any example herein, particularly any of examples 16 to 19, wherein the central lasso portion and the lateral lasso portions are formed from a tension member having two extreme end portions fastened together by a lock knot, and wherein the central lasso portion comprises the lock knot.

Example 21: A leaflet lasso according to any example herein, particularly any of examples 16 to 20, wherein the lateral lasso portions are spaced along the central lasso portion.

Example 22: A leaflet lasso according to any example herein, particularly any of examples 16 to 21, further comprising a tissue anchor retained on each lateral lasso portion.

Example 23: A leaflet lasso according to any example herein, particularly example 22, wherein each tissue anchor comprises an eyelet member and an anchor member, and wherein each lateral lasso portion extends through the eyelet member of the respective tissue anchor.

Example 24: A leaflet lasso according to any example herein, particularly example 22, wherein the tissue anchor is bioresorbable.

Example 25: A leaflet lasso according to any example herein, particularly any of examples 16 to 24, wherein the central lasso portion and the lateral lasso portions are bioresorbable.

Example 26: The leaflet lasso of any example herein, particularly any one of examples 16 to 25, wherein the central lasso portion comprises one or more radiopaque markers.

Example 27: A prosthetic device comprises an annular frame movable between a radially compressed configuration and a radially expanded configuration; a leaflet assembly disposed within the annular frame and coupled to the annular frame; and a leaflet lasso, which comprises a central lasso portion positioned over an end of the leaflet assembly, the central lasso portion defining a central opening; and at least one lateral lasso portion fastened to a leaflet of the leaflet assembly, the lateral lasso portion coupled to the central lasso portion such that radial expansion of the central lasso portion by applying tension to the central lasso portion results in axial contraction of the lateral lasso portion.

Example 28: A prosthetic device according to any example herein, particularly example 27, wherein the lateral lasso portion comprises two tie-in portions and an anchor portion, and further comprising a tissue anchor retained on the anchor portion.

Example 29: A prosthetic device according to any example herein, particularly example 27, wherein the lateral lasso portion comprises two tie-in portions and an anchor portion, and wherein the anchor portion extends through the leaflet such that the two tie-in portions are disposed on opposite sides of the leaflet.

Example 30: A prosthetic device according to any example herein, particularly any of examples 27 to 29, wherein the central lasso portion comprises a first loop, wherein the lateral lasso portion comprises a second loop, and wherein the first loop is coupled to the second loop by a slip knot.

Example 31: A prosthetic device according to any example herein, particularly example 27, wherein the central lasso portion and the lateral lasso portion are on different planes and form a single loop.

Example 32: A prosthetic device according to any example herein, particularly example 31, wherein the lateral lasso portion comprises two tie-in portions and an anchor portion, wherein the two tie-in portions are wound together to form an interwound portion, wherein a first end of the interwound portion is connected to the anchor portion, and wherein a second end of the interwound portion is connected to the central lasso portion.

Example 33: A prosthetic device according to any example herein, particularly any of examples 27 to 32, wherein the central lasso portion comprises a plurality of loops arranged to define the central opening.

Example 34: A prosthetic device according to any example herein, particularly any of examples 27 to 33, wherein the central lasso portion comprises one or more radiopaque markers.

Example 35: A prosthetic device according to any example herein, particularly any of examples 27 to 34, wherein the leaflet lasso is bioresorbable.

Example 36: A prosthetic device comprises an annular frame movable between a radially compressed configuration and a radially expanded configuration; a leaflet assembly disposed within the annular frame and coupled to the annular frame; and a leaflet lasso, which comprises a central lasso portion positioned over an end of the leaflet assembly, the central lasso portion defining a central opening; and a plurality of lateral lasso portions fastened to a plurality of leaflets of the leaflet assembly, each of the lateral lasso portions coupled to the central lasso portion such that radial expansion of the central lasso portion by applying tension to the central lasso portion results in axial contraction of each of the lateral lasso portions.

Example 37: A method comprises positioning a central lasso portion of a leaflet lasso over an end of a leaflet assembly of a heart valve; attaching a lateral lasso portion of the leaflet lasso to a leaflet of the leaflet assembly; and applying tension to the central lasso portion to radially expand the central lasso portion, wherein the lateral lasso portion axially contracts as the central lasso portion radially expands and folds the leaflet along an axial direction of the leaflet.

Example 38: A method of any example herein, particularly example 37, further comprises delivering an expandable prosthetic heart valve in a crimped state to a central region of the leaflet assembly; and radially expanding the prosthetic heart valve to implant the prosthetic heart valve within the central region of the leaflet assembly.

Example 39: A method of any example herein, particularly example 38, wherein delivering the expandable prosthetic heart valve in the crimped state to the central region of the leaflet assembly precedes applying tension to the central lasso portion.

Example 40: A method of any example herein, particularly example 39, wherein positioning the central lasso portion of the leaflet lasso over the end of the leaflet assembly of the heart valve comprises aligning a central opening defined by the central lasso portion with the central region; and positioning a portion of the expandable prosthetic heart valve within the central opening.

Example 41: A method of any example herein, particularly example 40, wherein positioning the portion of the prosthetic heart valve within the central opening comprises detecting one or more radiopaque markers in the central lasso portion.

Example 42: A method of any example herein, particularly any of examples 40 to 41, wherein applying tension to the central lasso portion comprises radially expanding the expandable prosthetic heart valve from the crimped state to a working diameter.

Example 43: A method of any example herein, particularly any of examples 37 to 42, further comprising constructing the leaflet lasso from a piece of string, wherein attaching the lateral lasso portion of the leaflet lasso to the leaflet of the leaflet assembly occurs during the construction of the leaflet lasso.

Example 44: A method of any example herein, particularly example 43, wherein attaching the lateral lasso portion of the leaflet lasso to the leaflet of the leaflet assembly comprises extending an anchor portion of the lateral lasso through the leaflet.

Example 45: A method of any example herein, particularly any of examples 37 to 42, wherein attaching the lateral lasso portion of the leaflet lasso to the leaflet of the leaflet assembly comprises attaching a tissue anchor retained on the lateral lasso portion to the leaflet.

Example 46: A method of any example herein, particularly example 45, wherein attaching the tissue anchor to the leaflet comprises inserting an anchor member of the tissue anchor through the leaflet such that the anchor member and the lateral lasso portion are disposed on opposite sides of the leaflet.

Example 47: A method of any example herein, particularly any of examples 37 to 46, wherein the leaflet assembly is a leaflet assembly of a native heart valve.

Example 48: A method of any example herein, particularly any of examples 37 to 46, wherein the leaflet assembly is a leaflet assembly of a prosthetic heart valve implanted within a native heart valve.

Example 49: A method of any example herein, particularly any of examples 37 to 48, further comprises attaching one or more additional lateral lasso portions of the leaflet lasso to one or more additional leaflets of the leaflet assembly, wherein each additional lateral lasso portion axially contracts as the central lasso portion radially expands and folds the respective leaflet along an axial direction of the leaflet.

Example 50: A method comprises installing a leaflet lasso on a leaflet assembly of a first prosthetic heart valve implanted within a native heart valve within a patient's body; positioning a second prosthetic heart valve in a crimped state within a central region of the leaflet assembly; and radially expanding the second prosthetic heart valve from the crimped state to a working diameter, wherein during at least a portion of radially expanding the second prosthetic heart valve the second prosthetic heart valve contacts a central lasso portion of the leaflet lasso and applies a radial force to the central lasso portion that results in folding of at least one leaflet of the leaflet assembly.

Example 51: The method according to any example herein, particularly example 50, wherein the leaflet is retained between the first prosthetic heart valve and the second prosthetic heart valve in the folded state after the second prosthetic heart valve has been expanded to the working diameter.

Example 52: The method according to any example herein, particularly example 50, wherein installing the leaflet lasso on the leaflet assembly of the first prosthetic heart valve comprises constructing the leaflet lasso outside of the patient's body and delivering the leaflet lasso to a location of the first prosthetic heart valve via the patient's vasculature.

Example 53: The method according to any example herein, particularly example 52, wherein installing the leaflet lasso further comprises attaching a lateral lasso portion of the leaflet lasso to the leaflet, and wherein the lateral lasso portion contracts to fold the leaflet when the radial force is applied to the central lasso portion.

Example 54: A method of any example herein, particularly any of examples 50 to 52, wherein the leaflet assembly comprises a plurality of leaflets, and wherein installing the leaflet lasso on the leaflet assembly comprises positioning the central lasso portion of the leaflet lasso over free edges of the leaflets; and attaching each of a plurality of lateral lasso portions of the leaflet lasso to one of the plurality of leaflets of the leaflet assembly, wherein each of the lateral lasso portions contracts to fold the leaflet to which the lateral lasso is attached when the radial force is applied to the central lasso portion.

Example 55: The method according to any example herein, particularly example 50, further comprising constructing the leaflet lasso simultaneously with installing the leaflet lasso on the leaflet assembly of the first prosthetic heart valve.

Example 56: The method according to any example herein, particularly example 50, wherein the leaflet assembly comprises a plurality of leaflets, and further comprising identifying the leaflet that is at risk of causing coronary artery obstruction prior to installing the leaflet on the leaflet assembly.

Example 57: A leaflet lasso for modifying a leaflet of a host heart valve comprises a central lasso portion defining a central opening; at least one lateral lasso portion coupled to the central lasso portion such that radial expansion of the central lasso portion by applying tension to the central lasso portion results in axial contraction of the lateral lasso portion, the lateral lasso portion comprising a first strand portion and a second strand portion; and a tissue anchor comprising a shell member having a cavity and a core member disposed at least partially within the cavity, wherein a first tail end of the first strand portion is coupled to the core member, and wherein the first tail end is releasable from the core member by axial movement of the core member relative to the shell member in a first direction.

Example 58: A leaflet lasso of any example herein, particularly example 57, wherein a portion of the first tail end extends around a circumference of the core member, and wherein the portion of the first tail end that extends around the circumference of the core member is removable from around the circumference of the core member by axial movement of the core member relative to the shell member in the first direction.

Example 59: A leaflet lasso of any example herein, particularly example 58, wherein the first tail end extends into the cavity through an opening in a wall of the shell member.

Example 60: A leaflet lasso of any example herein, particularly any of examples 57 to 59, further comprising a release member attached to the core member, wherein the core member is axially movable relative to the shell member by applying tension to the release member.

Example 61: A leaflet lasso of any example herein, particularly any of examples 57 to 60, wherein the shell member comprises first and second closed end portions, and wherein the core member is enclosed within the cavity and positioned between the first and second closed end portions.

Example 62: A leaflet lasso of any example herein, particularly example 61, further comprising a spring disposed between the core member and the first closed end portion, wherein the spring is compressible by axial movement of the core member relative to the shell member in the first direction.

Example 63: A leaflet lasso of any example herein, particularly any of examples 61 to 62, wherein a second tail end of the second strand portion is attached to the shell member.

Example 64: A leaflet lasso of any example herein, particularly example 63, wherein the second tail end extends into the cavity through an opening in a wall of the shell member.

Example 65: A leaflet lasso of any example herein, particularly any of examples 57 to 60, wherein the shell member comprises a closed end portion and an open end, and wherein the core member is disposed partially within the cavity such that a first end portion of the core member is disposed within the cavity and a second end portion of the core member protrudes from or is exposed at the open end of the shell member.

Example 66: A leaflet lasso of any example herein, particularly example 65, wherein the release member is attached to the first end portion of the core member and a second tail end of the second strand portion is attached to the second end portion of the core member.

Example 67: A leaflet lasso of any example herein, particularly example 66, wherein the second tail end extends into the cavity through a first opening in a wall of the shell member, passes through a second opening in the wall of the shell member, and extends along an exterior of the shell member and core member to an attachment point at the second end portion of the core member.

Example 68: A leaflet lasso of any example herein, particularly example 67, wherein the second strand portion comprises a stopper member that engages the shell member and limits a length of the second tail end extending between the first opening in the wall of the shell member and the attachment point at the second end portion of the core member.

Example 69: A method comprises fastening a leaflet lasso to a leaflet assembly of a first prosthetic heart valve implanted within a native heart valve within a patient's body using a tissue anchor coupled to the leaflet lasso; positioning a second prosthetic heart valve in a crimped state within a central region of the leaflet assembly; radially expanding the second prosthetic heart valve from the crimped state to a working diameter, wherein during at least a portion of radially expanding the second prosthetic heart valve the second prosthetic heart valve contacts a central lasso portion of the leaflet lasso and applies a radial force to the central lasso portion that results in folding of a leaflet of the leaflet assembly; detaching the leaflet lasso from the leaflet assembly by applying tension to a release member coupled to the tissue anchor; and removing the leaflet lasso from the patient's body.

Example 70: A method according to any example herein, particularly example 69, wherein the leaflet lasso comprises a central lasso portion and a lateral lasso portion coupled to the central lasso portion such that the central lasso portion and lateral lasso portion are mutually adjustable by applying tension to the central lasso portion, wherein a strand portion of the lateral lasso portion is coupled to the tissue anchor prior to fastening the leaflet lasso to the leaflet assembly, and wherein detaching the leaflet lasso from the leaflet assembly comprises detaching the strand portion from the tissue anchor.

Example 71: A method according to any example herein, particularly example 70, wherein detaching the leaflet lasso from the leaflet assembly by applying tension to the release member comprises axially displacing a core member of the tissue anchor relative to a shell member of the tissue anchor by the tension applied to the release member.

Example 72: A method according to any example herein, particularly example 71, wherein displacing the core member of the tissue anchor relative to the shell member of the tissue anchor comprises compressing a spring disposed between the core member and the shell member by the tension applied to the release member.

Example 73: A medical assembly comprises a handle; a tube having a first proximal end, a first distal end, and a first lumen extending from the first proximal end to the first distal end, the tube coupled to the handle and axially displaceable relative to the handle; a hollow needle having a second proximal end, a second distal end, and a second lumen extending from the second proximal end to the second distal end, the hollow needle disposed within the first lumen and axially movable within the first lumen and relative to the tube; and an implantable leaflet folding device coupled to the needle.

Example 74: A medical assembly according to any example herein, particularly example 73, wherein the tube comprises a first longitudinal slot extending from the first distal end towards the first proximal end, and wherein the first longitudinal slot is connected to the first lumen.

Example 75: A medical assembly according to any example herein, particularly example 74, wherein the hollow needle comprises a second longitudinal slot extending from the second distal end towards the second proximal end, and wherein the second longitudinal slot is connected to the second lumen.

Example 76: A medical assembly according to any example herein, particularly example 75, wherein the second longitudinal slot is radially aligned with the first longitudinal slot to form a passage that connects the second lumen to an exterior of the tube.

Example 77: A medical assembly according to any example herein, particularly any one of examples 73-76, wherein the hollow needle is shorter than the tube such that the hollow needle can be fully retracted within the tube.

Example 78: A medical assembly according to any example herein, particularly any one of examples 73-77, wherein the second distal end comprises a beveled end.

Example 79: A medical assembly according to any example herein, particularly any one of examples 73-78, further comprising a stabilization member attached to the tube, the stabilization member extending laterally outward from the tube.

Example 80: A medical assembly according to any example herein, particularly any one of examples 73-79, further comprising a first shaft coupled to the handle and the hollow needle, wherein the first shaft is axially displaceable relative to the handle to move the hollow needle axially relative to the tube.

Example 81: A medical assembly according to any example herein, particularly any one of examples 73-80, further comprising a second shaft coupled to the handle and the tube, wherein the second shaft is axially displaceable relative to the handle to move the tube axially relative to the hollow needle.

Example 82: A medical assembly according to any example herein, particularly any one of examples 73-81, further comprising a third shaft coupled to the handle, the third shaft axially displaceable relative to the handle to extend over the tube or be retracted from the tube.

Example 83: A medical assembly according to any example herein, particularly any one of examples 73-82, wherein the leaflet folding device comprises a lasso portion and an anchor member coupled to the lasso portion, wherein the lasso portion is configured to be implanted adjacent to or on coaptation edges of leaflets of a host valve, and wherein the anchor member is configured to be anchored to one of the leaflets of the host valve.

Example 84: A delivery accessory for a leaflet folding device comprises a tube having a first proximal end, a first distal end, and a first lumen extending from the first proximal end to the first distal end, the tube having a first longitudinal slot extending from the first distal end towards the first proximal end; and a hollow needle having a second proximal end, a second distal end, and a second lumen extending from the second proximal end to the second distal end, the hollow needle disposed within the first lumen and axially movable within the first lumen and relative to the tube, the hollow needle having a second longitudinal slot extending from the second distal end towards the first proximal end, the second longitudinal slot radially aligned with the first longitudinal slot to form a passage through which at least a portion of the leaflet folding device can extend into the second lumen.

Example 85: A delivery accessory of any example herein, particularly example 84, wherein the hollow needle is shorter than the tube such that the hollow needle can be fully retracted within the tube.

Example 86: A delivery accessory of any example herein, particularly any one of examples 84-85, wherein the second distal end comprises a beveled end.

Example 87: A delivery accessory of any example herein, particularly any one of examples 84-86, further comprising a stabilization member attached to the tube, the stabilization member extending laterally outward from the tube.

Example 88: A delivery accessory of any example herein, particularly example 87, wherein the stabilization member comprises a first stabilization arm and a second stabilization member attached to the tube at axially spaced locations.

Example 89: A leaflet folding device for a host heart valve comprises a lasso member comprising: a central lasso portion having a central axis; and a lateral lasso portion connected to the central lasso portion and radially offset from the central axis, wherein radial expansion of the central lasso portion axially contracts the lateral lasso portion; and an anchor member coupled to the lasso member, the anchor member comprising a first anchor radially offset from the central axis.

Example 90: A leaflet folding device of any example herein, particularly example 89, wherein the first anchor is radially offset from the lateral lasso portion.

Example 91: A leaflet folding device of any example herein, particularly any one of examples 88-89, wherein the anchor member further comprises a second anchor axially offset from the first anchor.

Example 92: A leaflet folding device of any example herein, particularly any one of examples 89-91, wherein the first anchor comprises an atraumatic tip.

Example 93: A leaflet folding device of any example herein, particularly any one of examples 89-91, wherein the first anchor comprises a sharp tip.

Example 94: A leaflet folding device of any example herein, particularly any one of examples 91-93, wherein the anchors are V-shaped anchors.

Example 95: A leaflet folding device of any example herein, particularly any one of examples 89-94, wherein the anchor member further comprises a bent portion connecting the lateral lasso portion to the anchor member, and wherein the bent portion radially offsets the first anchor relative to the lateral lasso portion.

Example 96: A leaflet folding device of any example herein, particularly any one of examples 89-95, wherein the central lasso portion defines a central opening, and wherein the lateral lasso portion comprises two vertical members spaced along a perimeter of the central opening.

Example 97: A leaflet folding device of any example herein, particularly example 96, wherein the lateral lasso portion further comprises a web extending between and connected to end portions of the two vertical members, and wherein the anchor member is connected to the web.

Example 98: A leaflet folding device of any example herein, particularly any one of examples 89-97, wherein one or both of the lasso member and the anchor member are made from a shape memory material.

Example 99: A leaflet folding device of any example herein, particularly example 98, wherein the shape memory material is Nitinol.

Example 100: A leaflet folding device of any example herein, particularly any one of examples 89-99, wherein the lasso member and the anchor member are integrally formed.

Example 101: A leaflet folding device for a host heart valve comprises a curved member having a first end portion and a second end portion spaced from each other; an anchor member; and a tether having a first tether portion extending between the first end portion and the second end portion and a second tether portion extending between the second end portion and the anchor member, wherein the tether is slidably coupled to the second end portion such that radial expansion of the curved member increases a length of the first tether portion and correspondingly decreases a length of the second tether portion.

Example 102: A leaflet folding device any example herein, particularly example 101, wherein an end portion of the first tether portion is fixedly attached to the first end portion, and wherein the tether passes through an opening in the second end portion.

Example 103: A leaflet folding device for a host heart valve comprises a guide element; a wire member looped through the guide element to define a central lasso portion and a lateral lasso portion, wherein a length of the central lasso portion and a length of the lateral lasso portion are mutually adjustable by sliding the wire member relative to the guide element; and an anchor member coupled to the lateral lasso portion.

Example 104: A leaflet folding device of any example herein, particularly example 103, wherein the guide element comprises a tube having a lumen and at least one opening formed in a wall of the tube and connected to the lumen, and wherein the wire member is looped through the lumen and the opening.

Example 105: A leaflet lasso, a prosthetic device, a medical assembly, a delivery accessory, or a leaflet folding device of any example herein, particularly any one of examples 1-104, wherein the leaflet lasso, the prosthetic device, the medical assembly, the delivery accessory, or the leaflet folding device is sterilized.

The subject matter has been described with a selection of implementations and examples, but these preferred implementations and examples are not to be taken as limiting the scope of the subject matter since many other implementations and examples are possible that fall within the scope of the subject matter. The scope of the claimed subject matter is defined by the claims.

1. A leaflet folding device for modifying a leaflet of a host heart valve, the leaflet folding device comprising:
a lasso member comprising:
a central lasso portion defining a central opening; and
a lateral lasso portion coupled to the central lasso portion such that radial expansion of the central lasso portion by applying tension to the central lasso portion results in axial contraction of the lateral lasso portion.

2. The leaflet folding device of claim 1, wherein the central lasso portion comprises a first loop, wherein the lateral lasso portion comprises a second loop, and wherein the first loop is coupled to the second loop by one or more slip knots.

3. The leaflet folding device of claim 1, wherein the central lasso portion and the lateral lasso portion are on different planes and form a single loop.

4. The leaflet folding device of claim 3, wherein the lateral lasso portion comprises two tie-in portions and an anchor portion, wherein first ends of the two tie-in portions are connected to the anchor portion, wherein second ends of the two tie-in portions are connected to the central lasso portion, and wherein the two tie-in portions are wound together to form an interwound portion.

5. The leaflet folding device of claim 1, wherein the central lasso portion comprises a plurality of loops arranged to define the central opening.

6. The leaflet folding device of claim 1, wherein the lasso member comprises one or more additional lateral lasso portions coupled to the central lasso portion such that radial expansion of the central lasso portion by applying tension to the central lasso portion results in axial contraction of the one or more additional lateral lasso portions.

7. The leaflet folding device of claim 1, further comprising:
a guide element; and
a wire member looped through the guide element to define the central lasso portion and the lateral lasso portion, wherein a length of the central lasso portion and a length of the lateral lasso portion are mutually adjustable by sliding the wire member relative to the guide element.

8. The leaflet folding device of claim 1, further comprising a tissue anchor coupled to the lateral lasso portion.

9. The leaflet folding device of claim 8, wherein the lateral lasso portion comprises a first strand portion and a second strand portion; and
wherein the tissue anchor comprises a shell member having a cavity and a core member disposed at least partially within the cavity, wherein a first tail end of the first strand portion is coupled to the core member, and wherein the first tail end is releasable from the core member by axial movement of the core member relative to the shell member in a first direction.

10. The leaflet folding device of claim 9, further comprising a release member attached to the core member, wherein the core member is axially movable relative to the shell member by applying tension to the release member.

11. The leaflet folding device of claim 10, wherein the shell member comprises first and second closed end portions;
wherein the core member is enclosed within the cavity and positioned between the first and second closed end portions;
wherein the tissue anchor further comprises a spring disposed between the core member and the first closed end portion; and
wherein the spring is compressible by axial movement of the core member relative to the shell member in the first direction.

12. The leaflet folding device of claim 8, wherein the tissue anchor comprises a first anchor portion radially offset from the lateral lasso portion, and wherein the first anchor portion comprises an atraumatic tip or a sharp tip.

13. The leaflet folding device of claim 12, wherein the tissue anchor comprises a second anchor portion axially offset from the first anchor portion.

14. The leaflet folding device of claim 11, wherein one or both of the lasso member and the tissue anchor are made from a shape memory material.

15. The leaflet folding device of claim 1, wherein the central lasso portion comprises one or more radiopaque markers.

16. A method comprising:
installing a leaflet folding device on a leaflet assembly of a first prosthetic heart valve implanted within a native heart valve;
positioning a second prosthetic heart valve in a crimped state within a central region of the leaflet assembly; and
radially expanding the second prosthetic heart valve from the crimped state to a working diameter, wherein during at least a portion of radially expanding the second prosthetic heart valve the second prosthetic heart valve contacts a central lasso portion of a lasso member of the leaflet folding device and applies a radial force to the central lasso portion that results in folding of at least one leaflet of the leaflet assembly.

17. The method of claim 16, wherein the leaflet is retained between the first prosthetic heart valve and the second prosthetic heart valve in the folded state after the second prosthetic heart valve has been expanded to the working diameter.

18. The method of claim 16, wherein the leaflet assembly comprises a plurality of leaflets, and wherein installing the leaflet folding on the leaflet assembly comprises:
positioning the central lasso portion of the lasso member of the leaflet folding device over free edges of the leaflets; and
attaching each of a plurality of lateral lasso portions of the lasso member to one of the plurality of leaflets of the leaflet assembly, wherein each of the lateral lasso portions contracts to fold the leaflet to which the lateral lasso portion is attached when the radial force is applied to the central lasso portion.

19. The method of claim 16, further comprising constructing the lasso member of the leaflet folding device simultaneously with installing the leaflet folding device on the leaflet assembly of the first prosthetic heart valve.

20. The method of claim 16, wherein installing the leaflet folding device on the leaflet assembly of the first prosthetic heart valve comprises:
constructing the leaflet folding device outside of a patient's body;
delivering the leaflet folding device to a location of the first prosthetic heart valve via the patient's vasculature; and
attaching a lateral lasso portion of the lasso member of the leaflet folding device to the leaflet, wherein the lateral lasso portion contracts to fold the leaflet when the radial force is applied to the central lasso portion.

21. The method of claim 16, wherein installing the leaflet folding device on the leaflet assembly of the first prosthetic heart valve comprises:
fastening the lasso member of the leaflet folding device to the leaflet assembly using a tissue anchor of the leaflet folding device;
detaching the lasso member from the leaflet assembly by applying tension to a release member coupled to the tissue anchor; and
removing the leaflet folding device from the patient's body.
